(19) United States
(12) Patent Application Publication
(10) Pub. No.: US 2025/0331834 A1
(43) Pub. Date: Oct. 30, 2025

(54) CARTRIDGE SYSTEM AND CARTRIDGE

(71) Applicant: OLYMPUS MEDICAL SYSTEMS CORP., Tokyo (JP)

(72) Inventors: Toshinori TAMURA, Hirosaki-shi (JP); Kazuhiro MORISAKI, Yokohama-shi (JP); Kotaro YAMADA, Tachikawa-shi (JP); Hiroshi MINAMI, Chofu-shi (JP)

(21) Appl. No.: 19/264,777

(22) Filed: Jul. 9, 2025

Related U.S. Application Data
(63) Continuation of application No. PCT/JP2024/001046, filed on Jan. 17, 2024.
(60) Provisional application No. 63/480,206, filed on Jan. 17, 2023.

(51) Int. Cl. A61B 17/00

(52) U.S. Cl. CPC A61B 17/0057; A61B 17/00234; A61B 2017/00296; A61B 2017/00584

(57) ABSTRACT

A cartridge system includes a clip unit including a clip and a connecting member having a connecting arm connectable to an applicator and a case having a storage area where the clip unit is movable along a longitudinal direction, wherein the case has an inner wall that suppresses swinging of the connecting arm in the storage area.

### CLAIM FOR PRIORITY

This application is a continuation application based on International Patent Application No. PCT/JP2024/001046 filed on Jan. 17, 2024, whose priority is claimed on U.S. Provisional Application No. 63/480,206, filed Jan. 17, 2023. Both of the content of the PCT International Application and the Japanese Application are incorporated herein by reference.

### BACKGROUND OF THE INVENTION

### Field of the Invention

The present invention relates to a cartridge system housing an endoscopic clip unit and a cartridge housing an endoscopic clip unit.

### Description of Related Art

In endoscopic treatment, a clip unit that can ligate a resected part after treatment to stop bleeding or the like is used. The clip unit includes a clip configured to pinch the resected part or the like, a pressing tube configured to house the clip and lock the clip in a closed state, and the like. The clip unit is introduced into a treatment position by a clip introduction device that can be inserted through a channel of an endoscope.

The clip unit is loaded into the clip introduction device using a cartridge (see, for example, PCT International Publication No. WO/2021/171407).

### SUMMARY OF INVENTION

In order to achieve the aforementioned objective, the present invention employs the following means.

According to a first aspect of the present invention, there is provided a cartridge system including: a clip unit including a clip and a connecting member having a connecting arm connectable to an applicator; and a case having a storage area where the clip unit is movable along a longitudinal direction, wherein the case has an inner wall that suppresses swinging of the connecting arm in the storage area.

According to a second aspect of the present invention, there is provided a cartridge capable of storing a clip unit including a clip and a connecting member having a connecting arm connectable to an applicator, the cartridge including: a case having a storage area where the clip unit is movable along a longitudinal direction, wherein the case has an inner wall that suppresses swinging of the connecting arm in the storage area.

### BRIEF DESCRIPTION OF THE DRAWINGS

FIG. 1 is a perspective view of a clip introduction device of a cartridge system according to a first embodiment.

FIG. 2 is a perspective view of a clip unit of the cartridge system.

FIG. 3 is a perspective view of the clip unit with a pressing member displayed transparently.

FIG. 4 is a cross-sectional view of the pressing member in a longitudinal direction.

FIG. 5 is a perspective view of a pressing tube of the pressing member.

FIG. 6 is a perspective view of the pressing tube when seen from a proximal end side.

FIG. 7 is a perspective view of a fastening member of the pressing tube.

FIG. 8 is a perspective view of a connecting member of the clip unit.

FIG. 9 is a view showing a distal end connecting portion in which a breaking portion breaks.

FIG. 10 is a view showing the distal end connecting portion connected to a clip.

FIG. 11 is a view showing the distal end connecting portion connected to the clip.

FIG. 12 is a cross-sectional view of the distal end connecting portion.

FIG. 13 is a perspective cross-sectional view of the distal end connecting portion.

FIG. 14 is a cross-sectional view of the clip unit loaded into the sheath of the clip introduction device.

FIG. 15 is a perspective view of a cartridge storing the clip unit.

FIG. 16 is a cross-sectional view of the cartridge storing the clip unit.

FIG. 17 is a cross-sectional view of the cartridge before the clip unit is stored.

FIG. 18 is a perspective view of a case without storing the clip unit.

FIG. 19 is a perspective view showing a locking portion of the case.

FIG. 20 is a front view of the case when seen in a direction of an arrow shown in FIG. 18.

FIG. 21 is a cross-sectional view of an inner wall along a longitudinal direction.

FIG. 22 is a cross-sectional view of the inner wall along a width direction.

FIG. 23 is a perspective view of a regulating member of the cartridge.

FIG. 24 is a plan view of the regulating member.

FIG. 25 is a view showing an intermediate-layer member of the regulating member grasped by a pair of arms of the clip unit.

FIG. 26 is an explanatory view of a method for loading the clip unit into the clip introduction device using the cartridge.

FIG. 27 is an explanatory view of a method for loading the clip unit into the clip introduction device using the cartridge.

FIG. 28 is an explanatory view of a method for loading the clip unit into the clip introduction device using the cartridge.

FIG. 29 is an explanatory view of a method for loading the clip unit into the clip introduction device using the cartridge.

FIG. 30 is an explanatory view of a method for loading the clip unit into the clip introduction device using the cartridge.

FIG. 31 is an explanatory view of a method for loading the clip unit into the clip introduction device using the cartridge.

FIG. 32 is an explanatory view of a method for loading the clip unit into the clip introduction device using the cartridge.

FIG. 33 is an explanatory view of a method for loading the clip unit into the clip introduction device using the cartridge.

FIG. 34 is an explanatory view of a method for loading the clip unit into the clip introduction device using the cartridge.

FIG. 35 is an explanatory view of a method for loading the clip unit into the clip introduction device using the cartridge.

FIG. 36 is an explanatory view of a method for loading the clip unit into the clip introduction device using the cartridge.

FIG. 37 is an explanatory view of a method for loading the clip unit into the clip introduction device using the cartridge.

FIG. 38 is an explanatory view of a method for loading the clip unit into the clip introduction device using the cartridge.

FIG. 39 is an explanatory view of a method for loading the clip unit into the clip introduction device using the cartridge.

FIG. 40 is an explanatory view of a method for storing the clip unit in the cartridge.

FIG. 41 is an explanatory view of a method for storing the clip unit in the cartridge.

FIG. 42 is an explanatory view of a method for storing the clip unit in the cartridge.

FIG. 43 is an explanatory view of a method for storing the clip unit in the cartridge.

FIG. 44 is an explanatory view of the clip unit loaded into the clip introduction device.

FIG. 45 is a view showing the clip unit introduced into a body.

FIG. 46 is a view showing the clip unit with the pair of arms closed.

FIG. 47 is a view showing the clip unit with the clip locked.

FIG. 48 is a view showing the clip unit from which the clip is separated.

FIG. 49 is a view showing a modification example of a pair of beam portions.

FIG. 50 is a view showing the modification example.

FIG. 51 is a perspective view of a connecting member of a cartridge system according to a second embodiment.

FIG. 52 is a perspective view of the connecting member when seen from the proximal end side.

FIG. 53 is a perspective view of the connecting member with the breaking portion broken.

FIG. 54 is a cross-sectional view showing the connecting member (residue) after the breaking portion is broken and a distal end side member is separated.

FIG. 55 is a cross-sectional view showing the connecting member (residue).

FIG. 56 is an enlarged view of an area surrounded by a dotted line in FIG. 55.

FIG. 57 is a cross-sectional view showing a state of the proximal end surface of the same connecting member.

FIG. 58 is a cross-sectional view showing a state of the proximal end surface.

FIG. 59 is a cross-sectional view showing a state of the proximal end surface.

FIG. 60 is a view showing a modification example of a proximal end opening.

FIG. 61 is a view showing the modification example.

### DESCRIPTION OF EMBODIMENTS

### First Embodiment

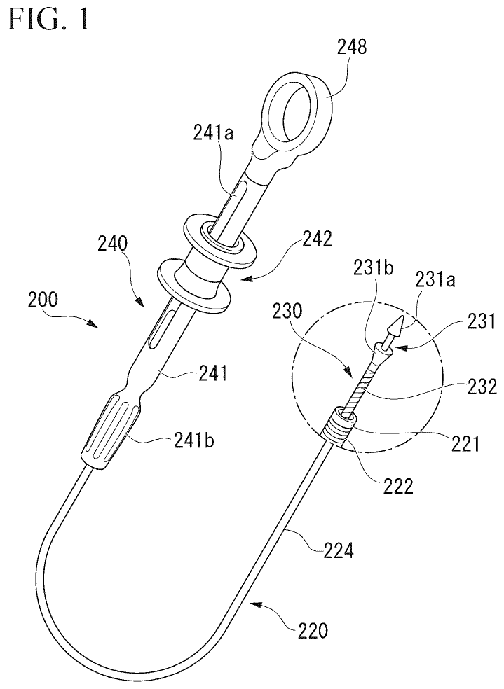

A cartridge system 100 according to a first embodiment of the present invention will be described with reference to FIGS. 1 to 48. The cartridge system 100 includes a clip unit 1 and a cartridge 5 configured to house the clip unit 1. The cartridge system 100 is a support system for easily loading the clip unit 1 into a clip introduction device 200.

### [Clip Introduction Device 200]

FIG. 1 is a perspective view of the clip introduction device 200.

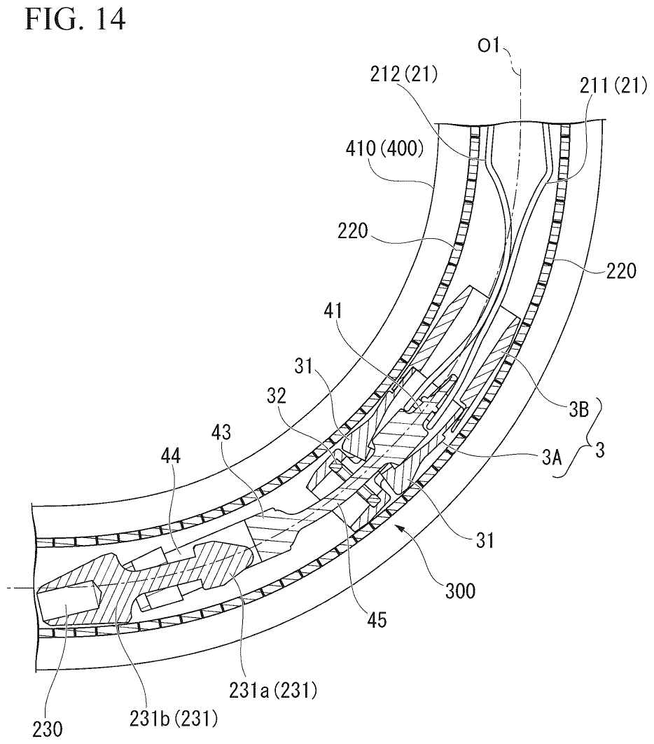

The clip introduction device (applicator) 200 includes a sheath 220, a manipulation wire 230, and a manipulation portion 240. The clip introduction device 200, for example, is inserted into a treatment tool insertion channel 410 of an endoscope 400 and used in combination with the endoscope 400 (FIG. 14). For this reason, the sheath 220 is formed to be sufficiently longer than the length of the treatment tool insertion channel 410 of the endoscope 400. The sheath 220 is flexible and bends in accordance with the curvature of an insertion portion of the endoscope 400.

The sheath 220 includes a distal end tip 221, a distal end side coil 222, and a proximal end side coil 224, and is formed in an elongated tubular shape as a whole. The distal end side coil 222 is arranged on the distal end side of the sheath 220. The distal end tip 221 is arranged at the distal end of the distal end side coil 222.

As shown in FIG. 1, the manipulation wire (motive power transmission portion) 230 includes an arrowhead hook (connecting portion) 231 connected to the clip unit 1 and a wire 232 configured to manipulate the arrowhead hook 231.

The arrowhead hook 231 includes a substantially conical engaging portion 231a configured to engage with the clip unit 1 and a wire connecting portion 231b provided at a proximal end of the engaging portion 231a. The arrowhead hook 231 is formed of a metallic material such as stainless steel.

The wire 232 is inserted to freely advance and retract with respect to the sheath 220. The distal end of the wire 232 is fixed to the proximal end of the wire connecting portion 231b by, for example, welding.

As shown in FIG. 1, the manipulation portion 240 includes a manipulation portion body 241, a slider 242, and a thumb ring 248. The manipulation portion body 241, the slider 242, and the thumb ring 248 are formed by injection molding using, for example, a resin material. The manipulation portion body 241 includes a slit portion 241a and a rotating grip 241b on the distal end side. The slit portion 241a supports the slider 242 so that advance and retraction are possible.

The slider 242 is movably attached in a longitudinal axis direction of the manipulation portion body 241, and has a proximal end of the wire 232 attached thereto. When the slider 242 advances and retracts along the manipulation portion body 241, the wire 232 advances and retracts with respect to the sheath 220, and the arrowhead hook 231 advances and retracts.

The thumb ring 248 is attached to the proximal end of the manipulation portion body 241 so that it is rotatable around the longitudinal axis of the manipulation portion body 241.

### [Clip Unit 1]

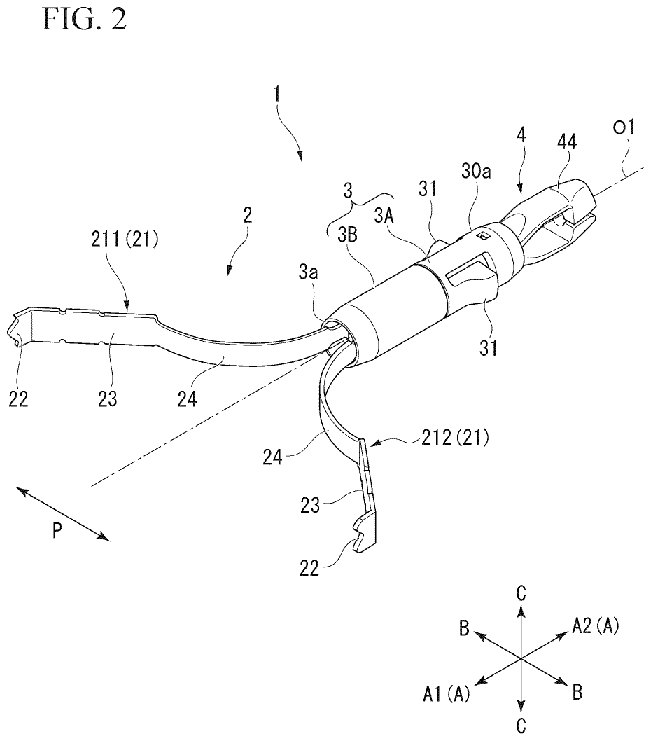

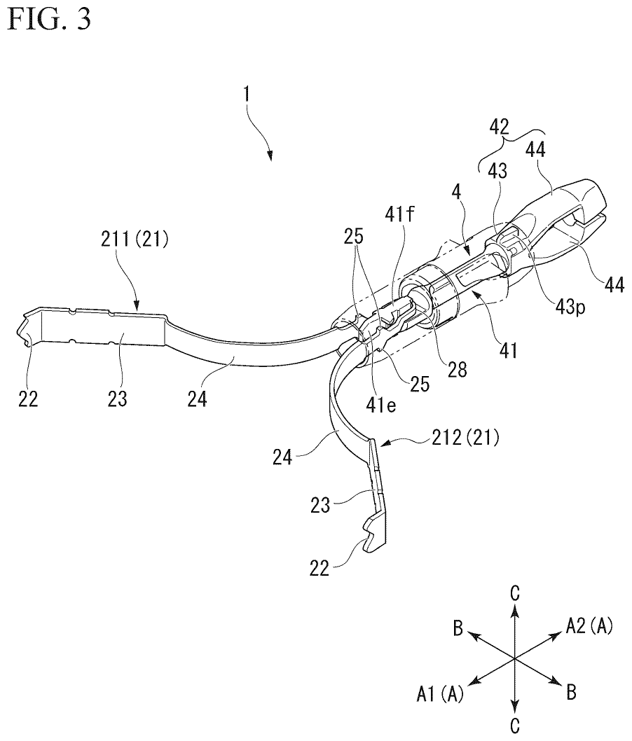

FIG. 2 is a perspective view of the clip unit 1. FIG. 3 is a perspective view of the clip unit 1 with the pressing member 3 displayed transparently. The clip unit 1 includes a clip 2, the pressing member 3, and a connecting member 4.

In the following description, the clip 2 side in the longitudinal direction A of the clip unit 1 is referred to as a distal end side (distal side) A1 of the clip unit 1 and the connecting member 4 side is referred to as a proximal end side (proximal side) A2 of the clip unit 1. Moreover, a direction perpendicular to the longitudinal direction A is referred to as a “second direction B” or a “left-right direction B.” Moreover, a direction perpendicular to the longitudinal direction A and the second direction B is referred to as a “first direction C” or an “up-down direction C.”

The clip (clip arm) 2 is formed by bending a metallic plate at the center. The clip 2 has a pair of arms 21 that can be opened and closed, and a proximal end portion 28 that connects the pair of arms 21. The proximal end side A2 of the clip 2 is inserted into an internal space 38 of the pressing member 3.

The pair of arms 21 include a first arm 211 and a second arm 212. The first arm 211 and the second arm 212 are arranged on both sides of a central axis O1 in the longitudinal direction A of the clip unit 1. In addition, the clip 2 may include three or more arms.

The first arm 211 has a tissue grasping portion 22, a flat plate-shaped grasping portion 23, a sliding portion 24, and an engaging portion 25 from the distal end side A1 toward the proximal end side A2. The tissue grasping portion 22 is formed by bending the distal end of the first arm 211 inward. The sliding portion 24 is a portion configured to elastically deform when the pair of arms 21 are pulled into the pressing member 3.

The second arm 212 has a tissue grasping portion 22, a flat plate-shaped grasping portion 23, a sliding portion 24, and an engaging portion 25 from the distal end side A1 toward the proximal end side A2.

The tissue grasping portion 22 of the first arm 211 and the tissue grasping portion 22 of the second arm 212 are formed in a shape asymmetrical to the central axis O1. Therefore, when the clip 2 is attached to the connecting member 4, the user can easily ascertain the orientation of the clip 2 to be attached to the connecting member 4. In addition, the tissue grasping portion 22 of the first arm 211 and the tissue grasping portion 22 of the second arm 212 may be formed in a symmetric shape with respect to the central axis O1.

The engaging portion 25 is a member that can engage with the fastening member 32 of the pressing member 3. The distal end side A1 of the engaging portion 25 is formed as a slope that forms an acute angle with respect to the longitudinal direction A and the proximal end side A2 is formed as a slope that forms an obtuse angle with respect to the longitudinal direction A.

The proximal end portion 28 connects the first arm 211 and the second arm 212. The first arm 211 and the second arm 212 connected by the proximal end portion 28 are provided so that they are freely opened and closed toward the distal end side A1. The proximal end portion 28 is bent in a U-shape and is connected to a hook 41f of the connecting member 4. The proximal end portion 28 is biased so that the pair of arms 21 are in an open state. Therefore, the pair of arms 21 of the clip 2 have a self-expanding force in an opening/closing direction P.

As shown in FIG. 3, the proximal end portion 28 and the hook 41f of the connecting member 4 are connected by inserting the hook 41f of the connecting member 4 into the proximal end portion 28 formed in the U-shape. When the proximal end portion 28 is connected to the connecting member 4, the opening/closing direction P of the pair of arms 21 approximately coincides with the left-right direction B.

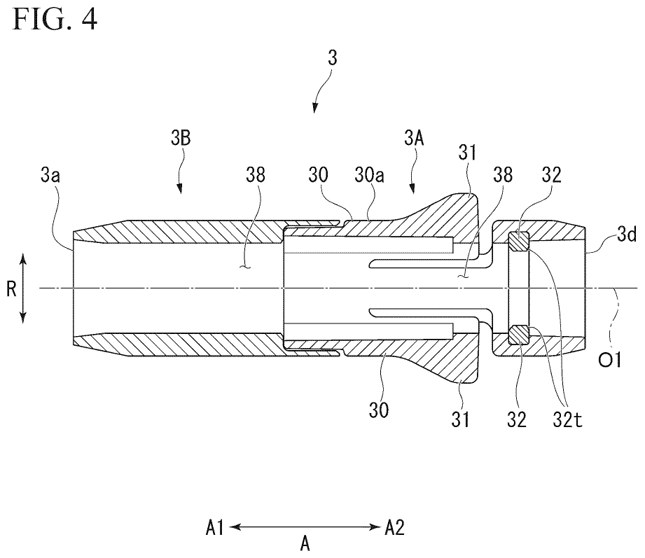

FIG. 4 is a cross-sectional view of the pressing member 3 in the longitudinal direction A.

The pressing member (tubular member) 3 is a cylindrical member capable of storing at least a part of the clip 2. The pressing member 3 has the internal space 38 where the clip 2 advances and retracts in the longitudinal direction A. The pressing member 3 can fix the clip 2 in a closed state after pulled into the internal space 38. The pressing member 3 has a pressing tube 3A provided on the proximal end side A2 and a pressing pipe 3B provided on the distal end side A1.

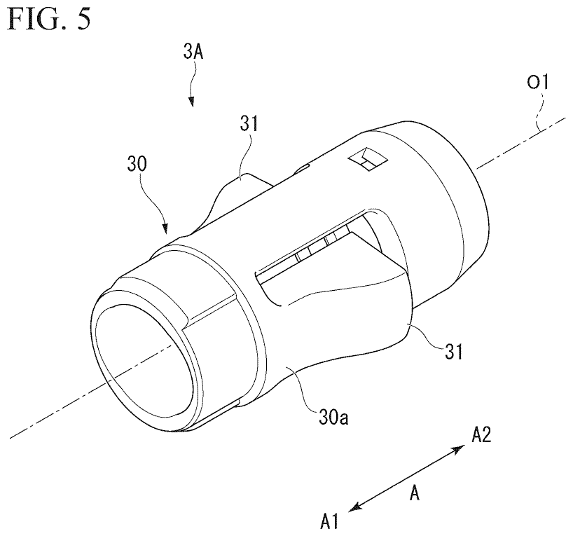

FIG. 5 is a perspective view of the pressing tube 3A.

The pressing tube (second tubular member) 3A has a pressing tube body 30 formed in a cylindrical shape, a retractable wing 31, and a fastening member 32.

The pressing tube body 30 is formed by performing an injection molding process on a material softer than the clip 2, for example, a thermoplastic resin having appropriate elasticity, such as polyphthalamide (PPA), polyamide (PA), polyether ether ketone (PEEK), or liquid crystal polymer (LCP). In addition, the pressing tube body 30 may be formed of metal instead of a thermoplastic resin.

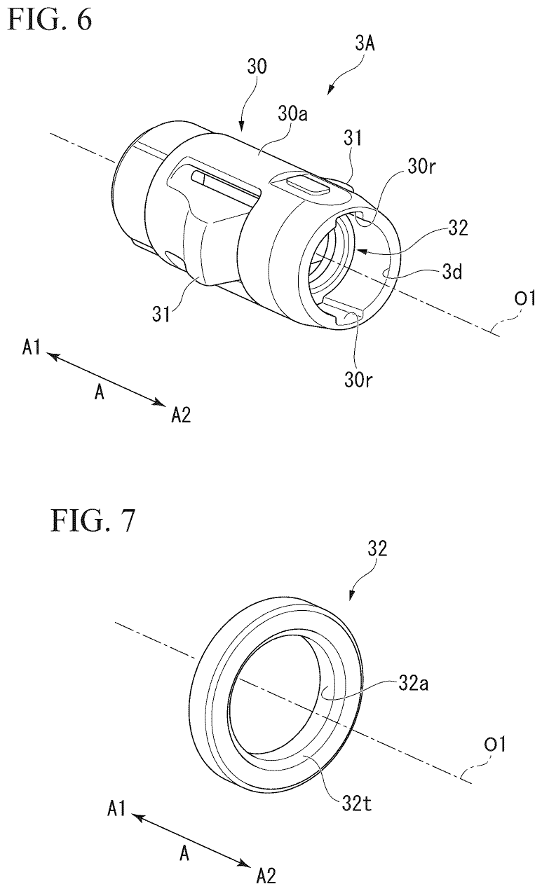

FIG. 6 is a perspective view of the pressing tube 3A when seen from the proximal end side A2.

An engaging concave portion 30r that engages with an engaging convex portion 43p of the connecting member 4 is formed in a proximal end opening 3d of the pressing tube body 30. An engaging concave portion 30r is provided on both sides of the central axis O1 in a state in which the central axis O1 is sandwiched therebetween.

The retractable wings 31 are a pair of convex portions that project and retract from an outer circumferential surface 30a of the pressing tube body 30. The retractable wings 31 are provided on both sides of the central axis O1 in the state in which the central axis O1 is sandwiched therebetween. The retractable wings 31 have a projected state in which they project outward in a radial direction R from the outer circumferential surface 30a as a basic posture. When the retractable wings 31 receive a force from the outside toward the inside in the radial direction R, they are put into a retracted state in which they retract into the outer circumferential surface 30a. When the above-described force is released, the retractable wings 31 return from the retracted state to the projected state.

The fastening member 32 is a ring-shaped member provided in the internal space 38 of the pressing tube 3A. The fastening member 32 is made of metal. In addition, the fastening member 32 only needs to be formed to be harder than the pressing tube body 30, and may be formed of, for example, a thermoplastic resin instead of metal.

The fastening member 32 is arranged so that the central axis of the fastening member 32 coincides with the central axis O1. The fastening member 32 is arranged on the proximal end side A2 of the retractable wing 31. The fastening member 32 is incorporated into the pressing tube body 30, for example, by insert molding. The fastening member 32 is arranged at a position projecting inward in the radial direction R from an inner circumferential surface of the pressing tube body 30.

FIG. 7 is a perspective view of the fastening member 32.

In the fastening member 32, a tapered surface 32t having an inner diameter decreasing from the proximal end side A2 toward the distal end side A1 on the proximal end side A2 is formed. Because the tapered surface 32t is provided on the fastening member 32, the connecting member 4 can be easily inserted into the fastening member 32 from the proximal end side A2.

The pressing pipe (first tubular member) 3B is a cylindrical member made of metal. The pressing pipe 3B is press-fitted into the distal end of the pressing tube 3A. The pressing tube 3A and the pressing pipe 3B may be connected by thermal welding, adhesion, or screw fastening.

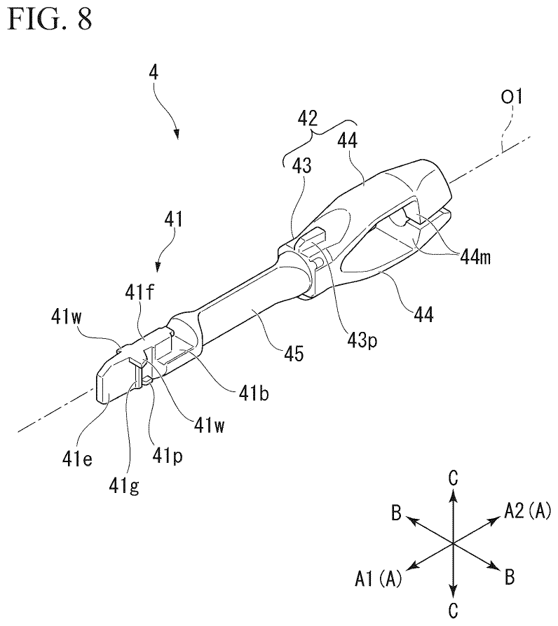

FIG. 8 is a perspective view of the connecting member 4.

The connecting member 4 is detachably connected to the proximal end portion 28 of the clip 2. Moreover, the connecting member 4 is detachably connected to the arrowhead hook 231 that passes through the sheath 220. That is, the connecting member 4 connects the clip 2 to the arrowhead hook 231. The connecting member 4 includes a distal end connecting portion 41 provided at the distal end, a proximal end connecting portion 42 provided at the proximal end, and a rod-shaped portion 45 that connects the distal end connecting portion 41 and the proximal end connecting portion 42.

The distal end connecting portion 41 has a hook 41f at the distal end, a breaking portion 41b provided on the proximal end side A2 of the hook 41f, a distal end expanding portion 41e, a first projection 41w, a second projection 41g, a third projection 41p, and a fourth projection 41q. The distal end connecting portion 41 can be inserted through the internal space 38 of the pressing member 3 in a state in which it is connected to the clip 2.

The hook 41f is a hook that extends in an up-down direction C perpendicular to the central axis O1 and is formed in an approximately cylindrical rod shape. The hook 41f extends from a “lower side” to an “upper side” in the up-down direction C. The proximal end portion 28 of the clip 2 is hooked onto the hook 41f from above.

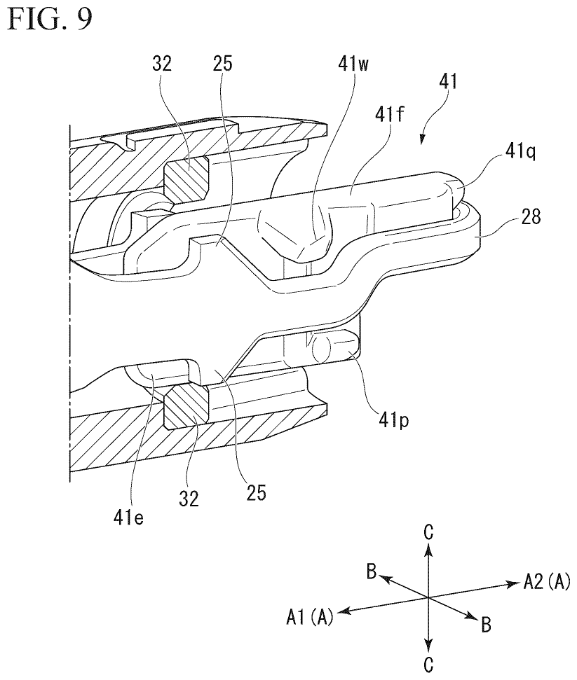

FIG. 9 is a view showing the distal end connecting portion 41 in which the breaking portion 41b has broken.

The breaking portion 41b breaks when the proximal end portion 28 is pulled toward the proximal end side A2, and a breaking force by pulling of, for example, 20 newtons (N) to 90 N, is applied to the hook 41f. In addition, the breaking portion 41b may have a mechanism for disconnecting the connection between the proximal end portion 28 of the clip 2 and the hook 41f of the connecting member 4. For example, the breaking portion 41b may have a mechanism for disconnecting the connection between the proximal end portion 28 and the hook 41f by performing deformation (plastic deformation or elastic deformation) without breaking.

The distal end expanding portion 41e is a member provided on the hook 41f and projecting toward the distal end side A1. The distal end expanding portion 41e has a tapered shape that narrows toward the distal end side A1 in both the up-down direction C and the left-right direction B.

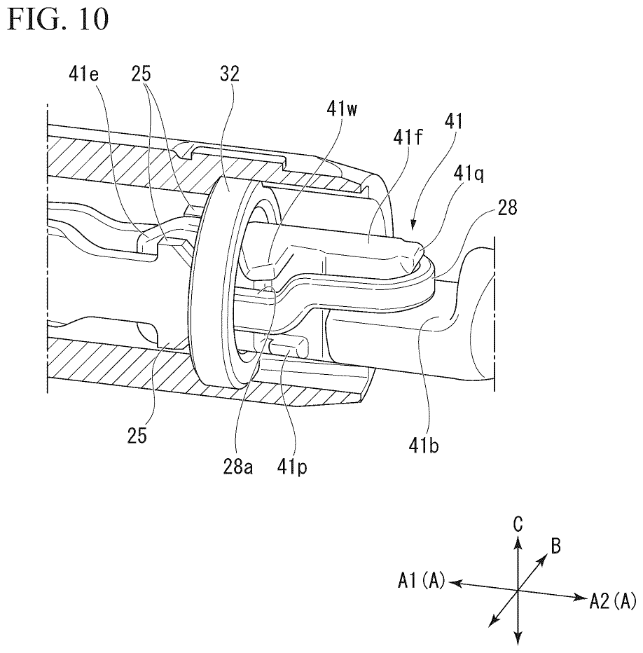

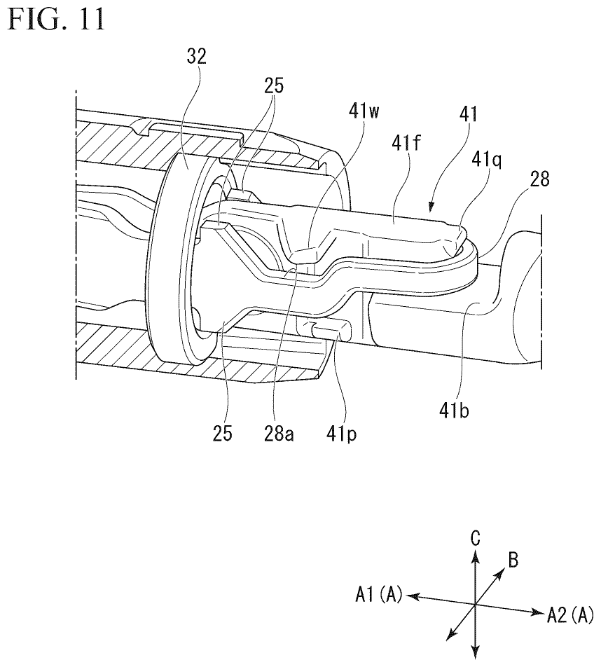

FIGS. 10 and 11 are views showing the distal end connecting portion 41 connected to the clip 2.

As shown in FIG. 10, when the pair of arms 21 are in the open state, the distal end of the distal end expanding portion 41e is arranged on the distal end side A1 of the engaging portion 25 of the clip 2 and is also arranged on the distal end side A1 of the fastening member 32. The distal end expanding portion 41e can come into contact with an inner circumferential surface 32a of the fastening member 32 in the up-down direction C. Therefore, the clip 2 connected to the distal end connecting portion 41 is less likely to rattle with respect to the distal end connecting portion 41 in the up-down direction C.

The first projection 41w is a member provided on the hook 41f and projecting in the left-right direction B. The first projection 41w is a pair of wing-shaped projections extending on both sides in the left-right direction B at the upper end of the hook 41f extending toward the upper side in the up-down direction C. The first projection 41w has a shape symmetrical to the central axis O1.

The first projection 41w is a member that presses an upper surface 28a of the clip 2 downward. In addition, it is only necessary for the first projection 41w to be any member pressing the clip 2 downward, for example, a member projecting from the hook 41f in the longitudinal direction A. In addition, the first projection 41w may be a member having elasticity and may be elastically deformed in the up-down direction C to press the clip 2 downward.

The first projection 41w overlaps at least a part of the upper surface 28a of the proximal end portion 28 of the clip 2. Therefore, the clip 2 connected to the distal end connecting portion 41 is less likely to rattle with respect to the distal end connecting portion 41 in the up-down direction C.

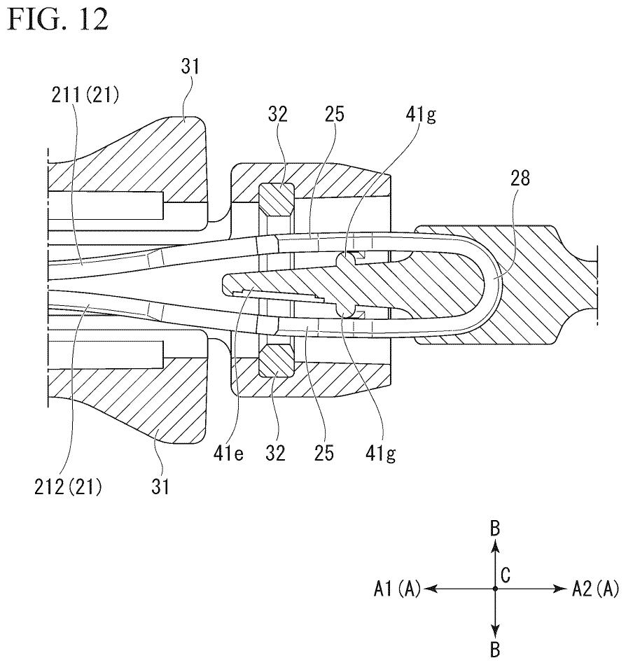

FIG. 12 is a cross-sectional view of the distal end connecting portion 41.

The second projection 41g is a member provided on the hook 41f and configured to project in the left-right direction B. The second projection 41g extends in the up-down direction C below the first projection 41w and is a pair of projections extending on both sides in the left-right direction B. The second projection 41g has a shape symmetrical to the central axis O1.

As shown in FIG. 11, when the proximal end portion 28 is pulled toward the proximal end side A2 of the pressing member 3, the pair of arms 21 are put into a closed state and the engaging portion 25 is pulled into the proximal end side A2 from the fastening member 32. When the engaging portion 25 is pulled into the proximal end side A2 from the fastening member 32, the engaging portion 25 engages with the fastening member 32. As a result, the clip 2 cannot move to the distal end side A1 and the pair of arms 21 are locked in the closed state. When the pair of arms 21 are locked in the closed state, the pair of arms 21 cannot return to the open state.

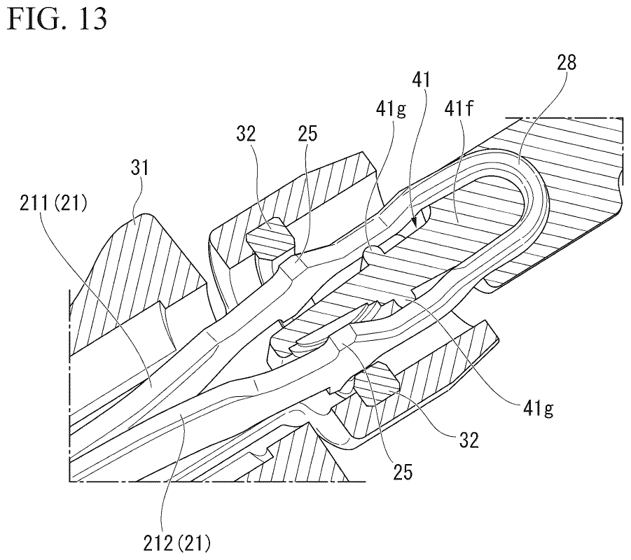

FIG. 13 is a perspective cross-sectional view of the distal end connecting portion 41.

When the pair of arms 21 deform so that the distance between the engaging portion 25 of the first arm 211 and the engaging portion 25 of the second arm 212 becomes smaller in the left-right direction B, the second projections 41g are arranged at positions where they abut against the pair of arms 21. Specifically, the second projection 41g is arranged on the proximal end side A2 of the engaging portion 25 in a state in which the engaging portion 25 is arranged on the distal end side A1 of the fastening member 32 and the engaging portion 25 abuts against the fastening member 32. Therefore, an amount of traction force required to lock the pair of arms 21 in the closed state is an appropriate amount of force instead of a small amount of force. As a result, it is possible to prevent the operator from unintentionally locking the pair of arms 21 in the closed state.

The third projection 41p is a member provided on the hook 41f and configured to project in the left-right direction B. The third projection 41p is a pair of projections extending on both sides in the left-right direction B at the lower end side of the hook 41f extending toward the upper side in the up-down direction C. The third projection 41p has a shape symmetrical to the central axis O1. The first projection 41w and the third projection 41p are arranged on both sides in the up-down direction C while sandwiching the pair of connected arms 21 therebetween.

As shown in FIG. 9, when the breaking portion 41b breaks, the third projection 41p is located in a fragment on the distal end side A1 of the breaking portion 41b. Even if the breaking portion 41b breaks, the third projection 41p maintains engagement with the pair of arms 21. Therefore, it is possible to prevent the fragment from falling off the pair of arms 21.

The fourth projection 41q is a member provided on the hook 41f and configured to project toward the proximal end side A2 in the longitudinal direction A. The fourth projection 41q is a projection that projects toward the proximal end side A2 at the upper end of the hook 41f extending toward the upper side in the up-down direction C. In the up-down direction C, the fourth projection 41q is arranged on the upper side of the proximal end portion 28 of the pair of connected arms 21. Therefore, it is possible to prevent the proximal end portion 28 of the pair of arms 21 from coming off the distal end connecting portion 41.

As shown in FIG. 8, the proximal end connecting portion 42 is an engaging portion with which the arrowhead hook 231 of the clip introduction device 200 is engaged (connected). The proximal end connecting portion 42 has a connecting portion body 43 and connecting arms 44.

The connecting portion body 43 is provided at the proximal end of the rod-shaped portion 45. The engaging convex portion 43p projecting in the up-down direction is formed on the connecting portion body 43. The engaging convex portion 43p engages with an engaging concave portion 30r provided in the proximal end opening of the pressing member 3, thereby regulating relative rotation between the pressing member 3 and the connecting member 4 in a circumferential direction with respect to the longitudinal direction A.

The connecting arms (a pair of legs) 44 are provided at the proximal end of the connecting portion body 43 and are bifurcated. The connecting arm 44 is elastically deformable in the up-down direction C with respect to the connecting portion body 43 and can be opened and closed with respect to the connecting portion body 43. A notch portion 44m for grasping and storing the engaging portion 231a of the arrowhead hook 231 is formed in the connecting arm 44. The notch portion 44m is formed in a shape that fits closely to the outer circumferential surface of the engaging portion 231a of the arrowhead hook 231. The connecting arm 44 can be connected to the arrowhead hook 231 of the clip introduction device 200 in an open state and can maintain the connection with the arrowhead hook 231 of the clip introduction device 200 in a closed state.

The rod-shaped portion 45 is an approximately rod-shaped member extending in the longitudinal direction A. The rod-shaped portion 45 is provided between the distal end connecting portion 41 and the proximal end connecting portion 42 and connects the distal end connecting portion 41 and the proximal end connecting portion 42. The rod-shaped portion 45 can be inserted through the internal space 38 of the pressing member 3.

The rod-shaped portion 45 is formed in an elliptical shape in which a first length in the left-right direction B is shorter than a second length in the up-down direction C in a cross-section perpendicular to the longitudinal direction A. The rod-shaped portion 45 may be formed in a rectangular or elliptical shape in which the first length is shorter than the second length in a cross-section perpendicular to the longitudinal direction A.

FIG. 14 is a cross-sectional view of the clip unit 1 loaded into the sheath 220.

The clip unit 1 loaded into the sheath 220 is introduced into a treatment position by passing through a treatment tool insertion channel 410 of the curved endoscope 400. The connecting member 4 has a rod-shaped portion 45 whose first length in the left-right direction B is shorter than its second length in the up-down direction C, and is more likely to bend in the left-right direction B than in the up-down direction C. The sheath 220 and the clip unit 1 loaded into the sheath 220 are also referred to as a clip device 300.

When the clip device 300 is inserted into the bending treatment tool insertion channel 410 of the endoscope 400, the clip unit 1 can easily pass through the bending treatment tool insertion channel 410 by aligning the curved direction of the treatment tool insertion channel 410 with the left-right direction B.

When the clip device 300 is bent in the left-right direction B, the engaging portion 231a of the arrowhead hook 231 can project from the proximal end connecting portion 42 in the left-right direction B. Therefore, the clip device 300 is easily bent in the left-right direction B.

### [Cartridge 5]

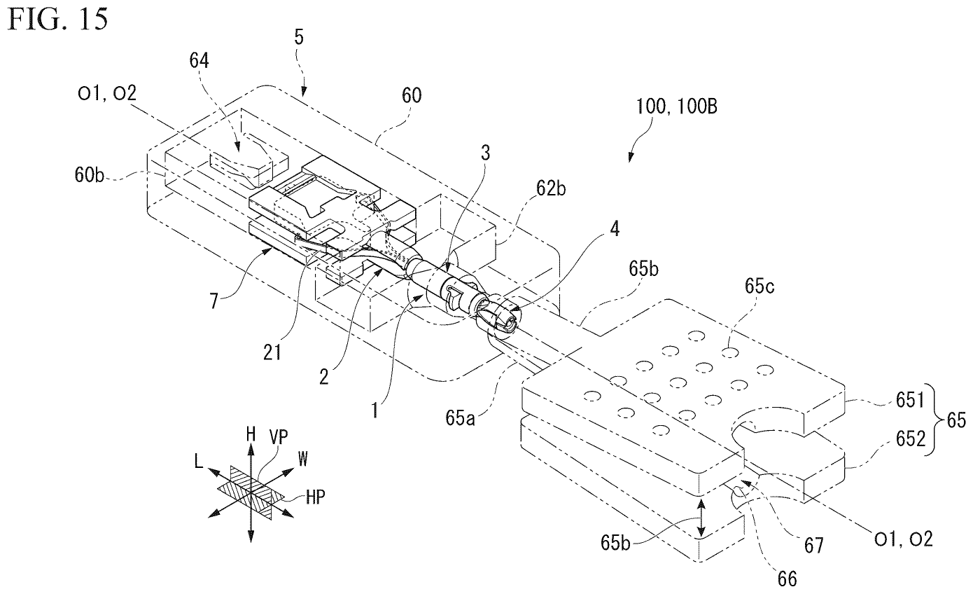

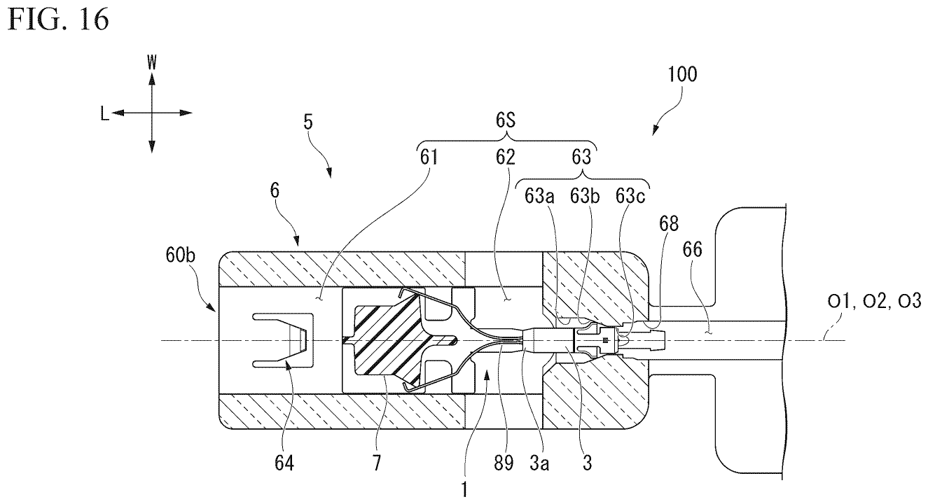

FIG. 15 is a perspective view of the cartridge 5 storing the clip unit 1. FIG. 16 is a cross-sectional view of the cartridge 5 storing the clip unit 1. The cartridge 5 has a case 6 and a regulating member 7. A length of the cartridge 5 in the longitudinal direction L is about 50 mm, a width is about 10 mm to 20 mm, and a thickness (height) is about 5 mm, so that the cartridge 5 is formed with a size that is easily held by hand.

As shown in FIG. 15, one of two directions perpendicular to the longitudinal direction L of the cartridge 5 and perpendicular to each other is defined as a “width direction W” and the other is defined as a “height direction H.” Moreover, a plane horizontal to the longitudinal direction L and the width direction W is defined as a “horizontal plane HP.” A plane horizontal to the longitudinal direction L and the height direction H is defined as a “vertical plane VP.” Moreover, in the cartridge 5 storing the clip unit 1, the pair of arms 21 side is defined as the distal end side of the cartridge 5 and the connecting member 4 side is defined as the proximal end side of the cartridge 5.

The case 6 has a case body 60, a compressing portion 65, and a sheath connecting portion 66. The case 6 is manufactured by injection molding from a transparent resin material having an appropriate hardness, such as ABS, PC, PP, PS, acrylic, or cycloolefin polymer. Because the case 6 is formed using a transparent resin material, it is easy for the user to determine whether or not the clip unit 1 is present inside.

The case body 60 is formed in a rectangular box shape. The length of the case body 60 in the width direction Wis longer than the length of the case body 60 in the height direction H.

In the case body 60, the storage area 6S in which the clip unit 1 is stored to be movable in the longitudinal direction L and the sheath insertion area 68 in which the sheath 220 is inserted are formed. The storage area 6S has a first area 61, a second area 62, and a folding area 63. As shown in FIG. 16, the first area 61, the second area 62, and the folding area 63 are arranged from the distal end to the proximal end in the longitudinal direction L. The first area 61, the second area 62, and the folding area 63 are internal spaces formed symmetrically with respect to a vertical plane VP including a central axis O2 of the cartridge 5 in the longitudinal direction L.

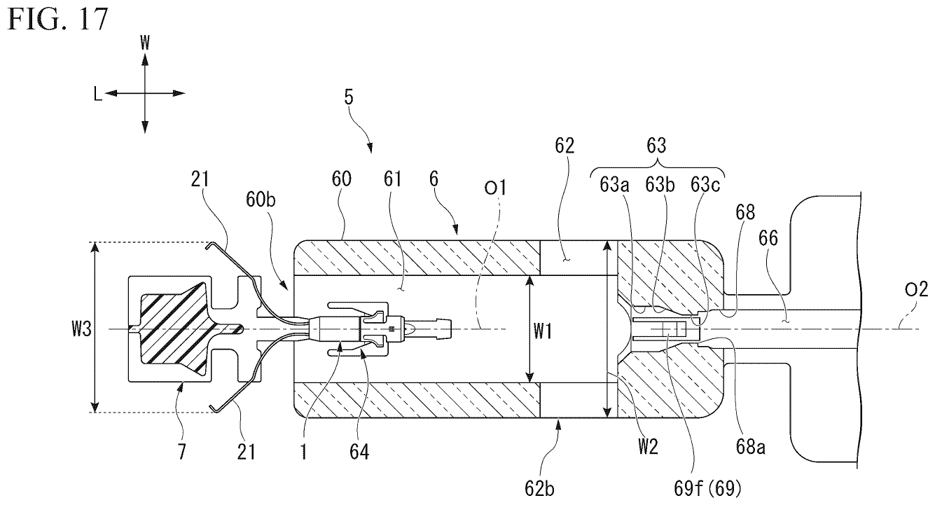

FIG. 17 is a cross-sectional view of the cartridge 5 before the clip unit 1 is stored.

The clip unit 1 is stored in the storage area 6S from a distal end opening 60b formed on the distal end side of the storage area 6S of the case body 60. The clip unit 1 is stored in the cartridge 5 from the connecting member 4 side.

As shown in FIG. 17, the clip unit 1 is stored in the storage area 6S when the central axis O1 of the clip unit 1 is aligned with the longitudinal direction L. The clip unit 1 is stored in the storage area 6S when the opening/closing direction P of the pair of arms 21 is aligned with the width direction W of the case 6.

The first area 61 is an internal space in which the clip unit 1 is stored to be movable in the longitudinal direction L. The first area 61 communicates with the second area 62. The first area 61 has a locking portion 64.

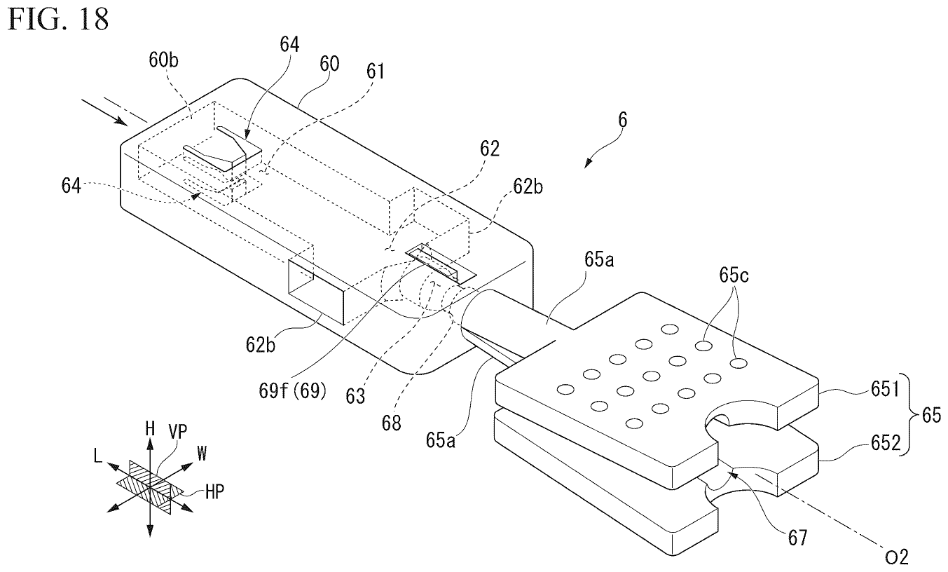

FIG. 18 is a perspective view of the case 6 without the clip unit 1 stored therein.

The locking portion 64 is the distal end side of the first area 61 and is formed on one side (upper side) and the other side (lower side) in the height direction H. The two locking portions 64 are formed symmetrically with respect to the horizontal plane HP including the central axis O2. In addition, the locking portion 64 may be formed on only one of the side (upper side) and the other side (lower side) of the first area 61 in the case body 60.

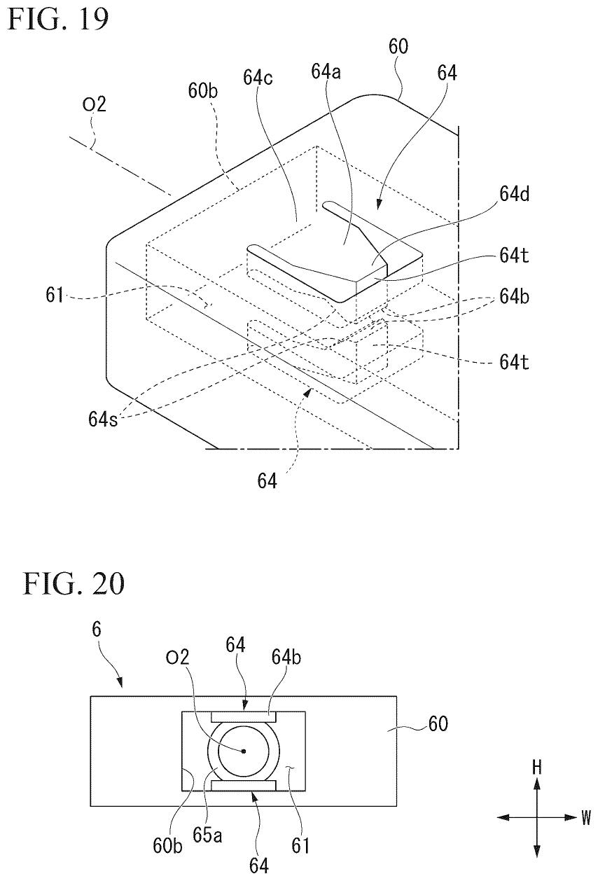

FIG. 19 is a perspective view showing the locking portion 64.

The locking portion 64 has an elastic deformation portion 64a and a locking convex portion 64b. The elastic deformation portion 64a is formed in a flat plate shape and only an end 64c on the distal end side in the longitudinal direction L is fixed to the case body 60. The elastic deformation portion 64a elastically deforms as a cantilever beam with the end 64c fixed to the case body 60 as the fixed end.

FIG. 20 is a front view of the case 6 when seen in the direction of the arrow shown in FIG. 18.

The locking convex portion 64b is provided on an end 64d of the elastic deformation portion 64a on the proximal end side in the longitudinal direction L. As shown in FIG. 20, the locking convex portion 64b projects toward the first area 61 side of the storage area 6S in a state in which no external force is applied (initial state). The locking convex portion 64b has an inclined surface 64s and a locking surface 64t.

The inclined surface 64s is formed on the distal end side of the locking convex portion 64b in the longitudinal direction L. The inclined surface 64s is inclined with respect to the central axis O2, and a normal line of the inclined surface 64s faces the distal end side in the longitudinal direction L.

The locking surface 64t is formed on the proximal end side of the locking convex portion 64b in the longitudinal direction L. The locking surface 64t is formed on a plane approximately perpendicular to the central axis O2 and the normal line of the inclined surface 64s faces the proximal end side in the longitudinal direction L.

The second area 62 is an internal space in which the clip unit 1 is movably stored in the longitudinal direction L. The length of the second area 62 in the longitudinal direction L is shorter than the length of the first area 61 in the longitudinal direction L. The second area 62 communicates with the folding area 63.

As shown in FIG. 17, a length W1 of the first area 61 in the width direction W is smaller than an open width W3 of the pair of arms 21 in the open state. Moreover, a length W2 of the second area 62 in the width direction W is larger than the open width W3 of the pair of arms 21 in the open state.

The second area 62 has openings 62b on both sides in the width direction W. The edges of the opening 62b are formed in a rectangular shape and extend in the height direction H and the longitudinal direction L.

The folding area 63 has an expanded diameter portion 63a, a tapered portion 63b, and a reduced diameter portion 63c. The expanded diameter portion 63a, the tapered portion 63b, and the reduced diameter portion 63c are arrayed from the distal end toward the proximal end.

The expanded diameter portion 63a is an area that allows the connecting arm 44 of the connecting member 4 to elastically expand (or to be opened or closed). In the expanded diameter portion 63a, when the arrowhead hook 231 of the clip introduction device 200 engages with the connecting member 4 of the clip unit 1, the connecting arm 44 of the connecting member 4 can be opened and closed in a direction perpendicular to the central axis O1.

The tapered portion 63b is provided on the proximal end side of the expanded diameter portion 63a and is formed in a tapered shape. The tapered portion 63b is enlarged in diameter from the proximal end side toward the distal end side. Therefore, when the pressing member 3 is slid from the distal end side toward the proximal end side, the retractable wings 31 of the pressing member 3 are stored inside the pressing tube body 30.

The reduced diameter portion 63c is an area where the retractable wing 31 is held in a retracted state. The reduced diameter portion 63c is an area where the connecting arm 44 of the connecting member 4 is not allowed to elastically expand (or to be opened or closed).

When the pressing member 3 is slid along the tapered portion 63b from the distal end side toward the proximal end side, the retractable wings 31 of the pressing member 3 are stored inside the pressing member 3. Therefore, the reduced diameter portion 63c smoothly connected to the tapered portion 63b can hold the retractable wings 31 of the pressing member 3 in a stored state (retracted state).

As shown in FIG. 15, the compressing portion 65 is a plate-shaped member provided at the proximal end of the case body 60. The compressing portion 65 has a first compressing portion 651 and a second compressing portion 652. The first compressing portion 651 and the second compressing portion 652 opposite to each other are provided in the height direction H of the case 6.

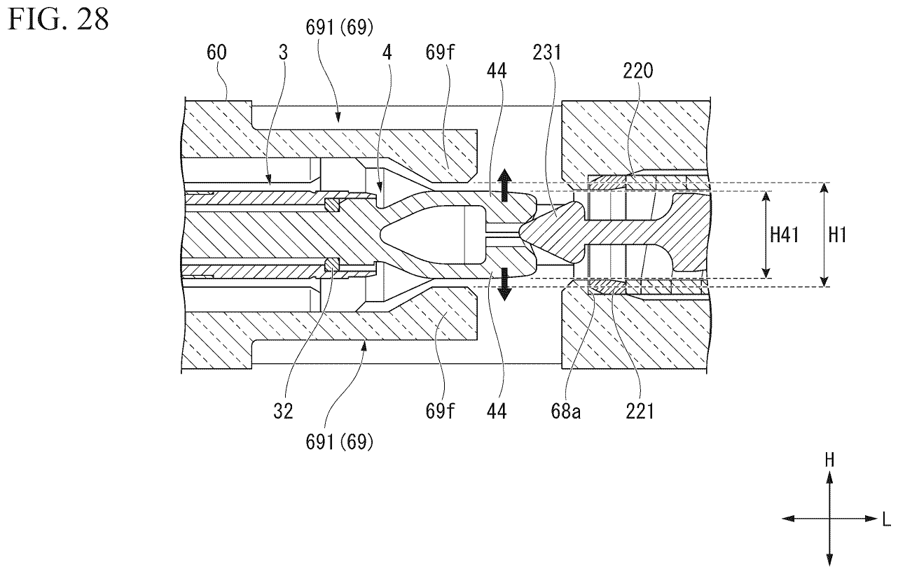

The sheath insertion area 68 is an area into which the distal end of the sheath 220 that has passed through the sheath connecting portion 66 is inserted. The sheath insertion area 68 is located on the proximal end side of the folding area 63 and communicates with the folding area 63. At the portion connected to the folding area 63, the length of the sheath insertion area 68 in the width direction W is longer than the length of the folding area 63 in the width direction W. In the sheath insertion area 68, a sheath abutment portion 68a against which the distal end tip 221 of the advanced sheath 220 can abut is formed (see FIG. 28).

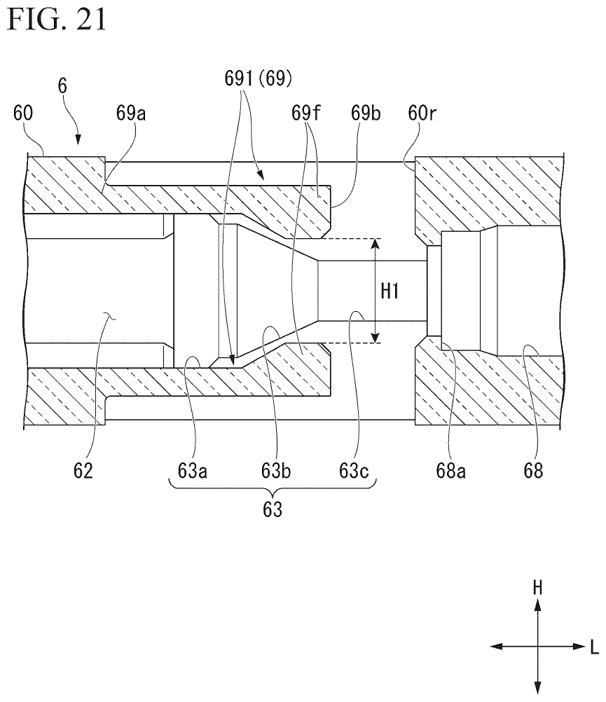

FIG. 21 is a cross-sectional view of the inner wall 69 along the longitudinal direction L.

An inner wall 69 is formed in the storage area 6S. The inner wall (swing regulating mechanism) 69 suppresses the swinging of the connecting arm 44. The inner wall 69 has a first inner wall 691 that suppresses the swinging of the connecting arm 44 in the height direction H and a second inner wall 692 that suppresses the swinging of the connecting arm 44 in the width direction W.

The first inner wall (vertical swing regulating mechanism) 691 is arranged at a position where engagement with the connecting member 4 of the clip unit 1 housed in the storage area 6S is possible. When the clip unit 1 abuts against the distal end in the longitudinal direction A in the storage area 6S, the first inner wall 691 and the connecting arm 44 are located to overlap in the longitudinal direction A. The first inner wall (vertical swing regulating mechanism) 691 has a pair of beam portions 69f provided on both sides in the height direction H.

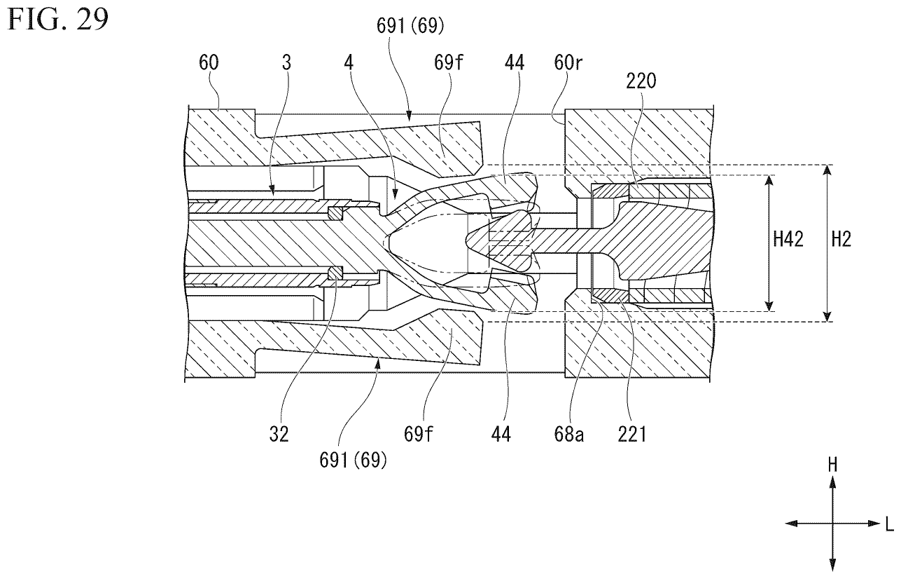

The pair of beam portions 69f are fixed to the proximal end side of the case body 60 and are cantilevers extending toward the proximal end side of the case body 60. A first end (fixed end) 69a of the beam portion 69f is fixed to the case body 60 and a second end (free end) 69b of the pair of beam portions 69f is spaced apart from the case body 60. The second end 69b projects inward in the height direction H compared to the first end 69a. The pair of beam portions 69f can transition between a first state (see FIG. 21) in which the length between the pair of beam portions 69f in the up-down direction C is a first length H1, and a second state (see FIG. 29) in which the length between the pair of beam portions 69f is a second length H2 longer than the first length H1. In the height direction H, the first length H1 between the pair of beam portions 69f is greater than an outer width H41 of the connecting arm 44 in the closed state (see FIG. 28).

The pair of beam portions (fingers) 69f are housed in a recess 60r provided in the case body 60. The recess 60r may be a through hole penetrating the case body 60 in the height direction H or may be a concave portion that does not penetrate the case body 60. Even if the pair of beam portions 69f elastically deform and transition to the second state, they do not project to the outside of the case body 60 in the height direction H. Therefore, the elastic deformation of the pair of beam portions 69f is not hindered by the fingers of the user who manipulates the case 6.

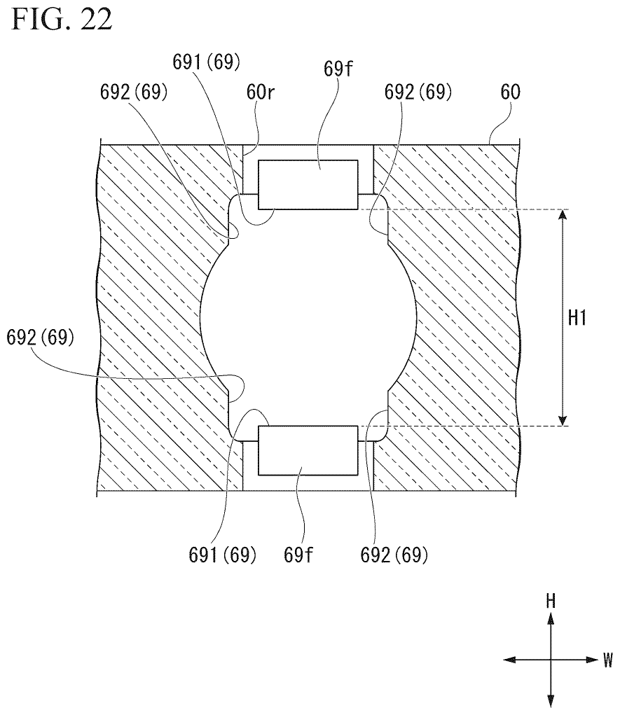

FIG. 22 is a cross-sectional view of the inner wall 69 along the width direction W.

The second inner wall (lateral swing regulating mechanism) 692 is an inner wall facing the width direction W and engages with the connecting arm 44 to suppress swinging of the connecting arm 44 in the width direction W. Two pairs of second inner walls 692 provided on both sides in the width direction W are arrayed in the height direction H.

The compressing portion 65 has a connecting portion 65a that connects to the case body 60. The connecting portion 65a separately connects the first compressing portion 651 and the second compressing portion 652 to the case body 60. The connecting portion 65a is bent so that the first compressing portion 651 and the second compressing portion 652 are spaced apart from each other. Therefore, a gap 65b is formed between the first compressing portion 651 and the second compressing portion 652. The first compressing portion 651 and the second compressing portion 652 are spaced apart further from each other on the proximal end side than on the distal end side.

The first and second compressing portions 651 and 652 are, for example, about 20 mm square, and are formed in a size suitable for pinching with fingers. For example, a plurality of semi-spherical concave portions 65c are formed on the outer surfaces of the first and second compressing portions 651 and 652 to prevent slipping during a compressing process.

The sheath connecting portion 66 is an insertion groove into which the sheath 220 can be inserted. The sheath connecting portion 66 is an arc-shaped groove formed on the inner surfaces of the first compressing portion 651 and the second compressing portion 652 and communicates with the sheath insertion area 68.

In a state in which the sheath 220 is inserted into the sheath connecting portion 66 from a proximal end opening 67, the user can fix the sheath 220 to the case 6 by performing the compressing process with the first compressing portion 651 and the second compressing portion 652.

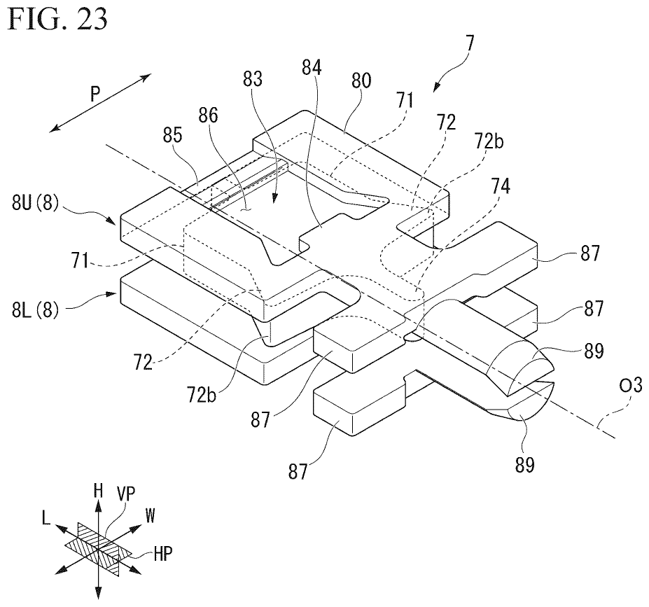

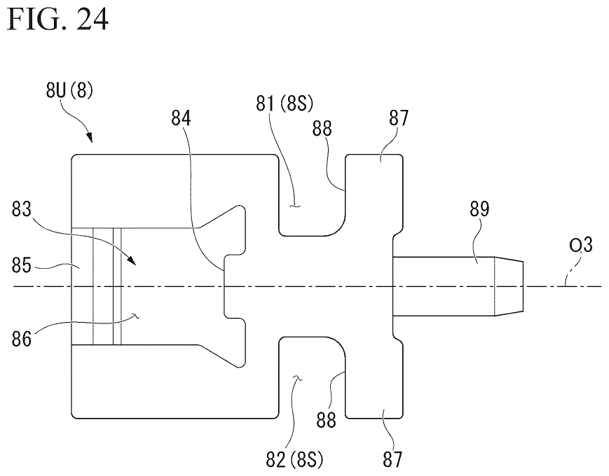

FIG. 23 is a perspective view of the regulating member 7. FIG. 24 is a plan view of the regulating member 7.

The regulating member (slider) 7 is movably stored in the first area 61 and the second area 62 together with the clip unit 1. The regulating member 7, for example, is formed from a resin similar to that of the case 6. The regulating member 7 does not have to be formed from a transparent resin like the case 6. As shown in FIG. 23, the regulating member 7 is formed in a shape symmetrical to the vertical plane VP and the horizontal plane HP including a central axis O3 of the regulating member 7 in the longitudinal direction L.

The regulating member 7 has an intermediate-layer member 70 and a guide member 8. The intermediate-layer member 70 has a distal end portion 71, a projected portion 72, a tapered portion 73, and a proximal end portion 74. The distal end portion 71, the projected portion 72, the tapered portion 73, and the proximal end portion 74 are arrayed from the distal end toward the proximal end along the central axis O3 direction of the regulating member 7.

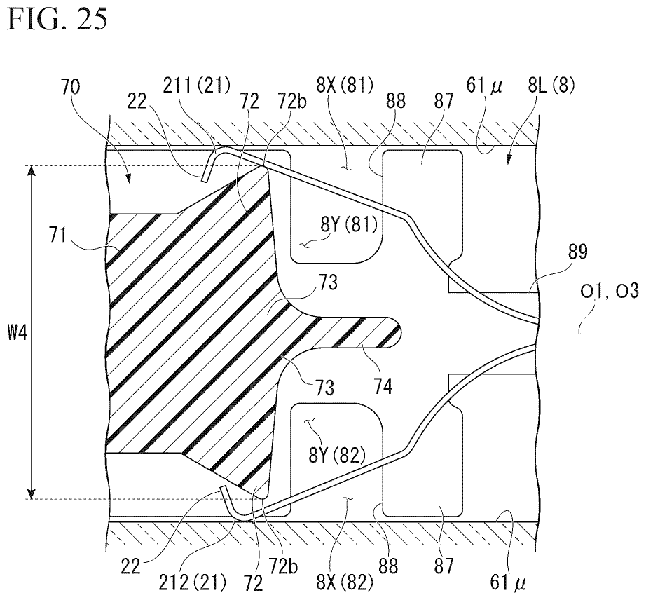

FIG. 25 is a view showing the intermediate-layer member 70 of the regulating member 7 grasped by the pair of arms 21.

The intermediate-layer member 70 is grasped by the clip 2 when the central axis O3 is substantially aligned with the central axis O1 of the clip unit 1. As shown in FIG. 16, the clip unit 1 is stored in the storage area 6S with the clip 2 grasping the regulating member 7. At this time, the central axis O1, the central axis O2, and the central axis O3 are desirably aligned with each other.

The distal end portion 71 is provided to project toward the distal end side from the projected portion 72. The distal end of the distal end portion 71 is located on a distal end side of the tissue grasping portion 22 of the clip 2 that grasps the regulating member 7. Moreover, the distal end of the distal end portion 71 is formed on a plane perpendicular to the central axis O3.

The projected portion 72 is a member that projects in a direction (hereinafter, also referred to as a “projecting direction P”) substantially perpendicular to the central axis O3. The projected portion 72 is provided on both sides of the central axis O3. The projected portion 72 is grasped by a first arm 211 and a second arm 212. The opening/closing direction P of the pair of arms 21 that grasp the projected portion 72 is substantially aligned with the projecting direction P of the projected portion 72. The first arm 211 and the second arm 212 are formed in a shape asymmetrical to the central axis O1. Therefore, as shown in FIG. 25, the first arm 211 and the second arm 212 that grasp the projected portion 72 are arranged at a position asymmetric to the central axis O3.

In the projected portion 72, a portion of a maximum projection from the central axis O3 in the projecting direction P is defined as a maximum projecting point 72b. A length W4 between maximum projecting points 72b is slightly smaller than a length W1 of the first area 61 in the width direction W. Therefore, the regulating member 7 is maintained in a state in which it is grasped by the pair of arms 21 in the first area 61.

The tapered portion 73 is a member formed in a tapered shape. The tapered portion 73 is provided on both sides of the central axis O3. The length of the tapered portion 73 in the projecting direction P is shorter than that of the projected portion 72. The length of the tapered portion 73 in the projecting direction P becomes shorter from the distal end side toward the proximal end side.

The proximal end portion 74 is a member formed to project from the tapered portion 73 to the proximal end side.

The guide member 8 adjusts a position of the regulating member 7 in the height direction H so that the projected portion 72 is grasped by the pair of arms 21. Moreover, the guide member 8 guides the regulating member 7 so that the regulating member 7 moves in the first area 61 along the longitudinal direction L. The guide member 8 has an upper-layer guide member 8U provided on one side (upper side) in the height direction H and a lower-layer guide member 8L provided on the other side (lower side) in the height direction H. The intermediate-layer member 70 is sandwiched between the upper-layer guide member 8U and the lower-layer guide member 8L in the height direction H. The upper-layer guide member 8U and the lower-layer guide member 8L are formed in a shape symmetrical to the horizontal plane HP including the central axis O3.

The upper-layer guide member 8U has an engaging portion 80, a wing portion 87, and a pressing portion 89. The engaging portion 80, the wing portion 87, and the pressing portion 89 are arrayed along the central axis O3 direction of the regulating member 7 from the distal end toward the proximal end.

In the engaging portion 80 of the upper-layer guide member 8U, an engaging concave portion 83 is formed on one side (upper side) in the height direction H. The engaging concave portion is a concave portion that engages with the locking convex portion 64b of the locking portion 64.

The engaging concave portion 83 is formed in a substantially rectangular shape when seen from above in the height direction H. An area of the engaging portion 80 where the engaging concave portion 83 is not formed is formed in a U-shape when seen from above.

As shown in FIG. 23, the engaging concave portion 83 has a locking surface 84 on the proximal end side in the longitudinal direction L that engages with the locking convex portion 64b of the locking portion 64. The locking surface 84 is formed on a plane approximately perpendicular to the central axis O3. On the other hand, the engaging concave portion 83 has a notch portion 85 formed on the distal end side in the longitudinal direction L. Therefore, a concave space 86 of the engaging concave portion 83 communicates with the space on the distal end side in the longitudinal direction L.

When the regulating member 7 moves in the first area 61 from the proximal end side to the distal end side, the locking surface 64t of the locking portion 64 passes through the notch portion 85 and the concave space 86 and abuts against the locking surface 84.

The wing portions 87 are provided on the proximal end side of the engaging portion 80, and are a pair of wing-shaped members extending on both sides in the width direction W. As shown in FIG. 24, the length of the wing portions 87 in the width direction W extending on both sides in the width direction Wis substantially equal to the length of the engaging portion 80 in the width direction W. A notch portion 88 notched toward an inward side in the width direction W is formed between the wing portions 87 and the engaging portion 80. The notch portion 88 forms a penetrating space 8S penetrating the upper-layer guide member 8U in the height direction H.

As shown in FIG. 25, when seen from above in the height direction H, at least a part of the pair of arms 21 grasping the projected portion 72 of the intermediate-layer member 70 overlaps the penetrating space 8S formed by the notch portion 88. When seen from above in the height direction H, the penetrating space 8S is divided into an outer penetrating space 8X located outside the pair of arms 21 in the width direction W, and an inner penetrating space 8Y located inside the pair of arms 21 in the width direction W.

When seen from above in the height direction H, the penetrating space 8S overlapping at least a part of the first arm 211 grasping the projected portion 72 of the intermediate-layer member 70 is also referred to as a first penetrating space 81. When seen from above in the height direction H, the penetrating space 8S overlapping at least a part of the second arm 212 grasping the projected portion 72 of the intermediate-layer member 70 is also referred to as a second penetrating space 82.

The pressing portion 89 is a member that regulates a minimum approach distance between the clip 2 and the pressing member 3. The pressing portion 89 is provided on the proximal end side of the wing portion 87. As shown in FIG. 16, because the pressing portion 89 engages with the edge of a distal end opening 3a of the pressing member 3, it is not possible to enter the internal space of the pressing member 3 from the distal end opening 3a. Therefore, even if the clip 2 is pulled in a direction in which the clip 2 approaches the pressing member 3, the pressing portion 89 regulates a minimum approach distance between the clip 2 and the pressing member 3 by engaging with the edge of the distal end opening 3a.

Like the upper-layer guide member 8U, the lower-layer guide member 8L has an engaging portion 80, a wing portion 87, and a pressing portion 89. The lower-layer guide member 8L is formed in a shape symmetrical to the upper-layer guide member 8U with respect to the horizontal plane HP including the central axis O3.

The regulating member 7 is stored in the first area 61 in a state in which the regulating member 7 is grasped by the pair of arms 21. As shown in FIG. 17, the length W1 of the first area 61 in the width direction W is smaller than the open width W3 of the pair of arms 21 in the open state. Therefore, the pair of arms 21 grasp the regulating member 7 when they are closed from the open state. The open width of the pair of arms 21 is approximately the length W1 of the width direction W of the first area 61. The pair of arms 21 abut against the case body 60 in the opening/closing direction P.

### [Method for Loading Clip Unit 1]

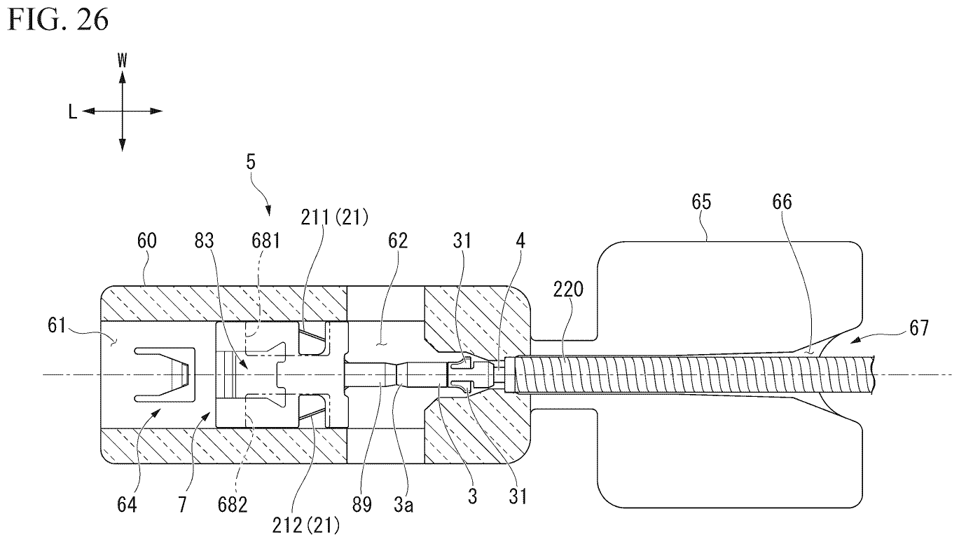

Next, a function of the cartridge system 100 will be described. FIGS. 26 to 39 are explanatory views of a method for loading the clip unit 1 into the clip introduction device 200 using the cartridge 5.

As shown in FIG. 26, the user inserts the sheath 220 of the clip introduction device 200 into the sheath connecting portion 66 of the case 6 from the proximal end opening 67. The user compresses the sheath 220 with the compressing portion 65 and fixes the sheath 220 to the case 6.

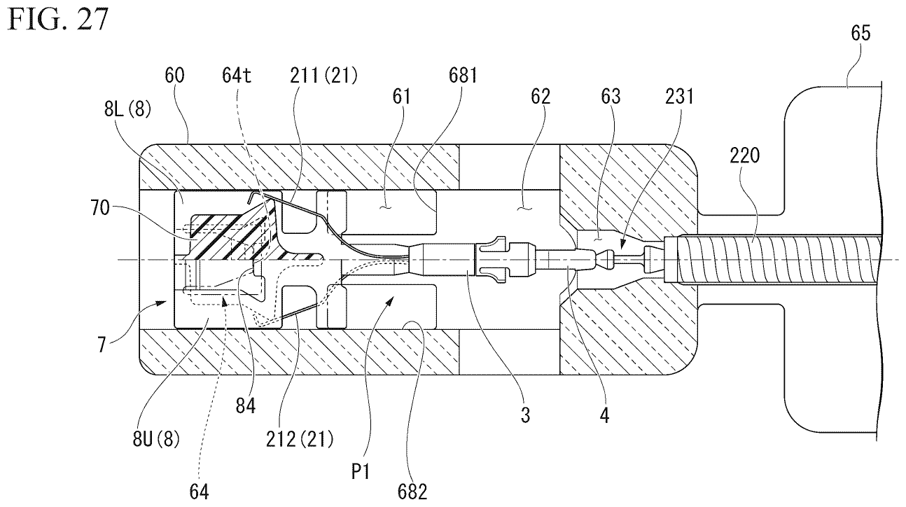

As shown in FIG. 27, the user manipulates the manipulation portion 240 to advance the manipulation wire 230 to the sheath 220, thereby advancing the arrowhead hook 231. The arrowhead hook 231 advances the connecting member 4, thereby advancing the regulating member 7 and the clip unit 1. As the regulating member 7 moves from the proximal end side to the distal end side, the locking surface 84 of the regulating member 7 abuts against and engages with the locking surface 64t of the locking portion 64. As a result, the clip unit 1 cannot advance toward the distal end side of its position. In other words, the locking portion 64 regulates the passage of the clip unit 1 moving toward the distal end side.

As shown in FIG. 28, the arrowhead hook 231 that is advanced engages with the connecting arm 44 of the connecting member 4 of the clip unit 1 in the height direction H. The arrowhead hook 231 elastically deforms the connecting arm 44 with respect to the height direction H and spreads the connecting arm 44 in the height direction H.

As shown in FIG. 29, when the connecting arms 44 are opened, the pair of beam portions 69f receive an external force from the connecting arms 44 and are deformed to be warped outward in the height direction H. Moreover, in a state in which the connecting arms 44 are closed, the beam portions 69f are arranged in close proximity to the connecting arms 44 and come into contact with the connecting member 4, thereby suppressing the swinging of the connecting member 4 in the height direction H.

It is only necessary for the pair of beam portions 69f to have a length sufficient to cover at least a part of the connecting arm 44. Thereby, the pair of beam portions 69f can come into contact with the outer diameter portion of the connecting arm 44, thereby suppressing swinging of the connecting member 4 in the height direction H.

In the height direction H, a second length H2 between the pair of beam portions 69f in the second state can be greater than an outer width H42 of the connecting arm 44 in the open state. The arrowhead hook 231 that is advanced pushes the connecting arm 44 apart until a gap that allows the arrowhead hook 231 to be inserted therein is created.

Even if the pair of beam portions 69f are subjected to an external force from the connecting arm 44 and are elastically deformed, they do not project outward from the case body 60 in the height direction H. Therefore, the elastic deformation of the pair of beam portions 69f is not hindered by the fingers of the user manipulating the case 6.

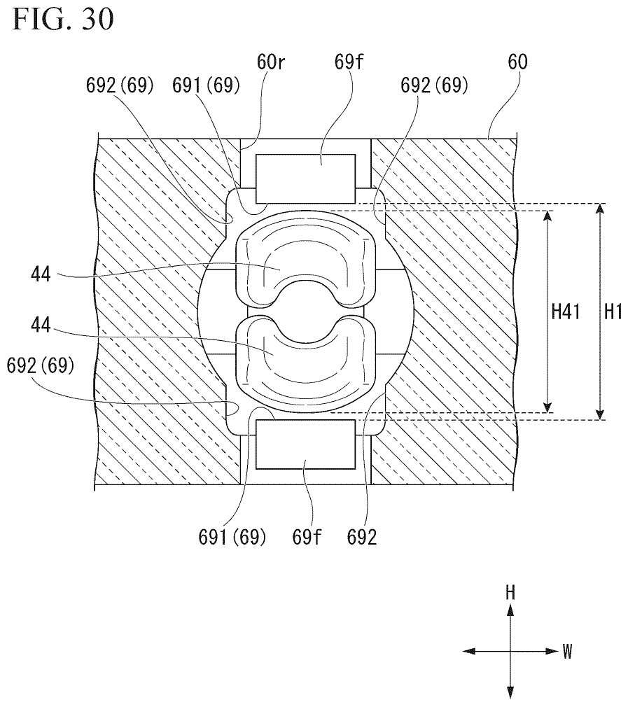

As shown in FIG. 30, the second inner wall 692 engages with the connecting arm 44 to suppress swinging of the connecting arm 44 in the width direction W.

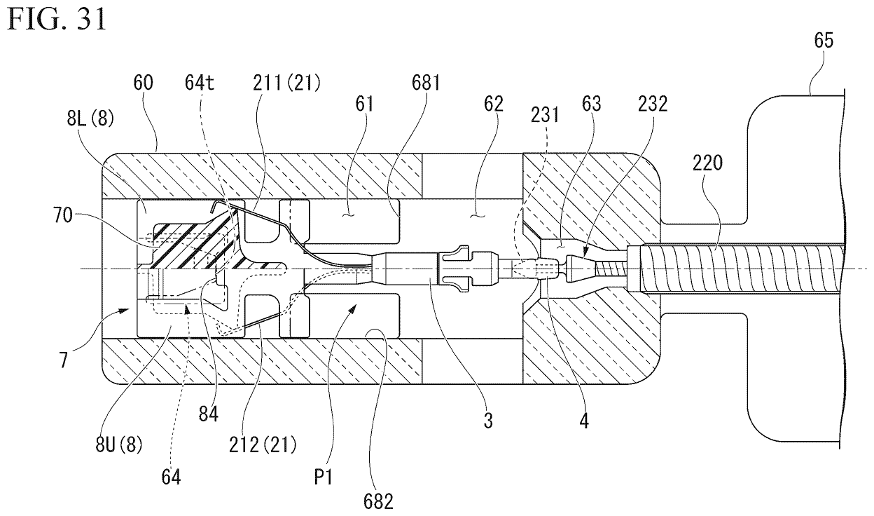

As shown in FIG. 31, the arrowhead hook 231 that is advanced is sandwiched between the connecting arms 44 of the connecting member 4 of the clip unit 1 and connected to the connecting member 4.

The locking surface 84 of the regulating member 7 is formed in a plane substantially perpendicular to the central axis O3. Moreover, the locking surface 64t of the locking portion 64 is formed in a plane substantially perpendicular to the central axis O2. Therefore, the regulating member 7 moving from the proximal end side to the distal end side is unlikely to push the locking portion 64 aside toward one side (upper side) in the height direction H. Moreover, even if the locking surface 84 of the regulating member 7 abuts against and engages with the locking surface 64t of the locking portion 64, the central axis O1 of the clip unit 1 is unlikely to deviate from the central axis O2 of the storage area 6S.

Even if the regulating member 7 does not have the engaging concave portion 83, the regulating member 7 moves from the proximal end side to the distal end side, thereby abutting against and engaging with the locking surface 64t of the locking portion 64. As a result, the arrowhead hook 231 that is advanced is connected to the connecting member 4 as described above.

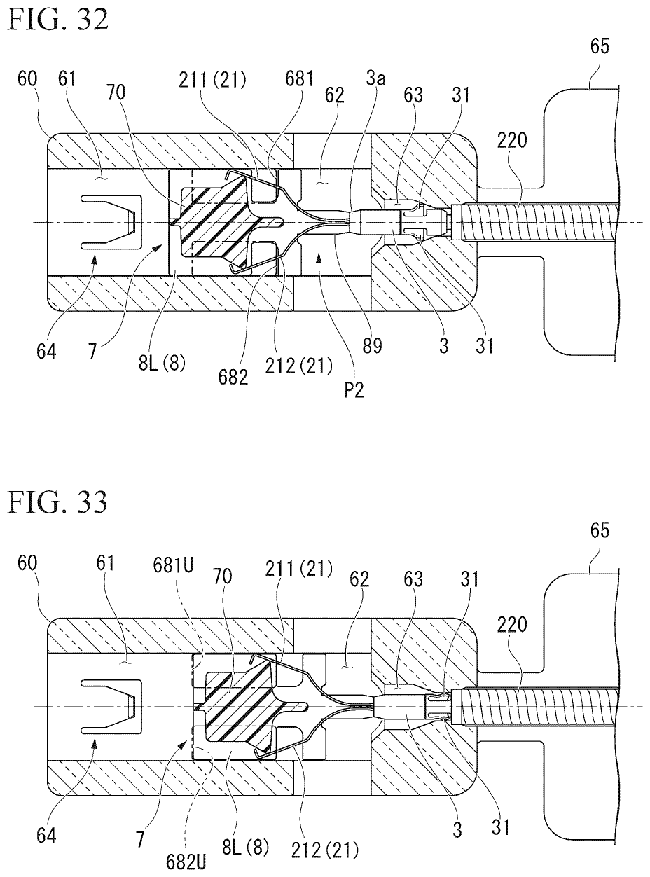

As shown in FIG. 32, the user pulls the manipulation wire 230. The clip 2 of the clip unit 1 is pulled toward the proximal end side by the connecting member 4 connected to the arrowhead hook 231. The hook 41f of the connecting member 4 pulls the proximal end portion 28 of the clip 2 without breaking. The regulating member 7 moves in the first area 61 of the storage area 6S in contact with the pair of arms 21. Because the pressing portion 89 of the regulating member 7 engages with the edge of the distal end opening 3a of the pressing member 3, the pressing member 3 is also pulled toward the proximal end side together with the clip 2.

The pair of arms 21 abut against the case body 60 in the opening/closing direction P in the first area 61. A frictional force generated by the abutment between the pair of arms 21 and the case body 60 makes it possible to preferably prevent the clip 2 from being pulled into the internal space of the pressing member 3 and being locked in the closed state by the pressing member 3.

When the clip 2 is pulled toward the proximal end side, the pressing portion 89 of the regulating member 7 engages with the edge of the distal end opening 3a of the pressing member 3, thereby regulating the minimum approach distance between the clip 2 and the pressing member 3. The pressing portion 89 abuts against the pressing member 3 in contact with the pair of arms 21 due to the pulling of the manipulation wire 230, and regulates the relative movement of the regulating member 7 with respect to the pressing member 3. This also makes it possible to preferably prevent the clip 2, which is pulled toward the proximal end side by the connecting member 4, from being pulled into the internal space of the pressing member 3 and being locked in the closed state by the pressing member 3.

The user pulls the clip unit 1 further toward the proximal end side. As shown in FIG. 33, the pressing member 3 passes through the folding area 63. The pressing member 3 is slid along the reduced diameter portion 63c from the distal end side toward the proximal end side, and the retractable wings 31 of the pressing member 3 are stored inside the pressing tube body 30. The pressing member 3, with the retractable wings 31 stored inside the pressing tube body 30, is pulled into the sheath 220.

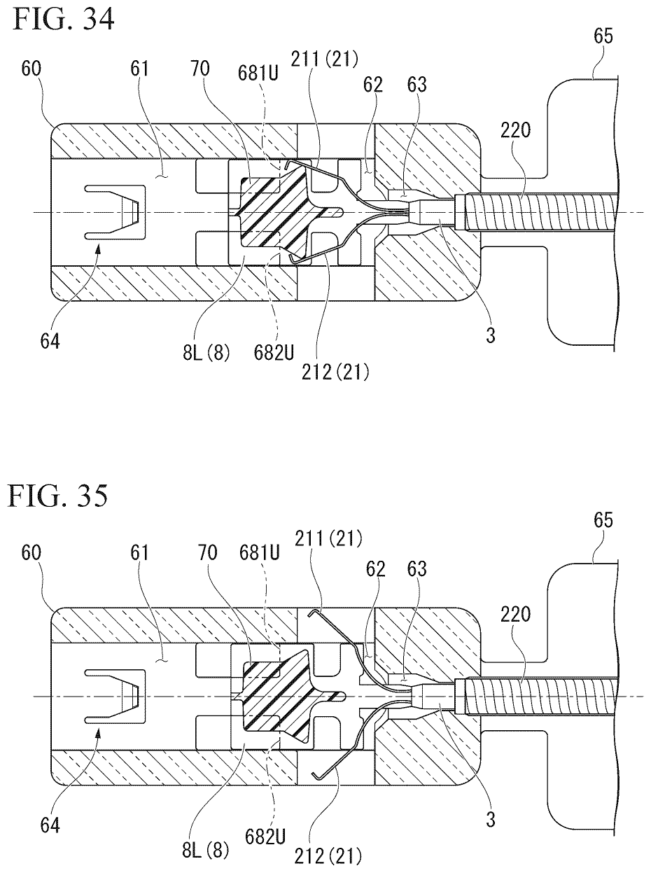

As shown in FIG. 34, when the retractable wings 31 of the pressing member 3 are stored inside the pressing member 3, contact points between the pair of arms 21 and the case body 60 are located in the first area 61. In other words, until the retractable wings 31 are stored inside the pressing member 3, the pair of arms 21 grasp the regulating member 7 and are not pulled into the internal space of the pressing member 3.

As shown in FIG. 35, the user further pulls the manipulation wire 230 to pull the regulating member 7 to the second area 62. The length W2 of the second area 62 in the width direction W is larger than the open width W3 of the pair of arms 21 in the open state. Therefore, the regulating member 7 is not grasped by the pair of arms 21 in the second area 62.

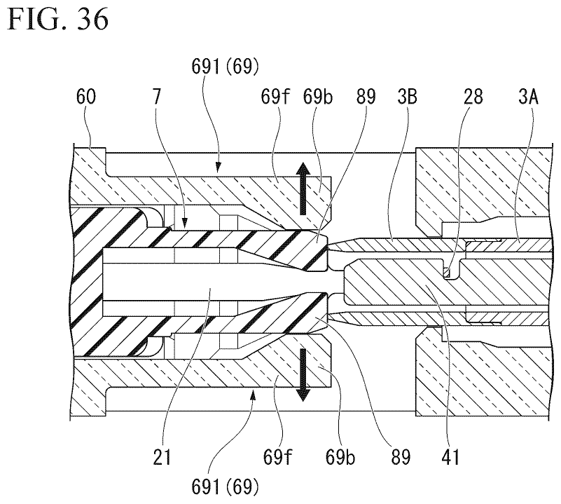

As shown in FIG. 36, the pressing portion 89 of the regulating member 7 is elastically deformed outward in the height direction H by contacting the pair of arms 21. At this time, the pressing portion 89 pushes the pair of beam portions 69f outward in the height direction H.

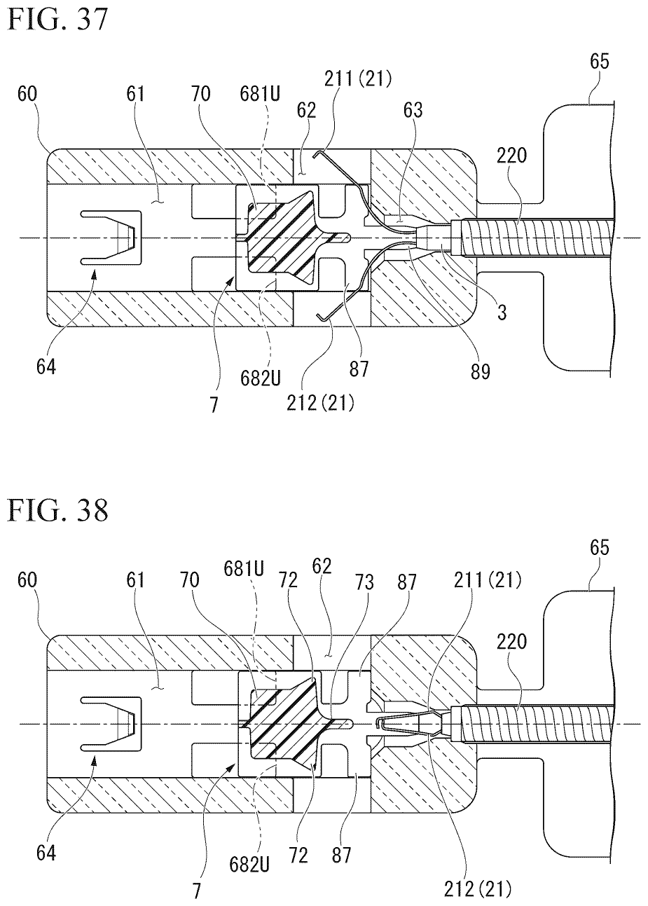

As shown in FIG. 37, the user further pulls the manipulation wire 230. The wing portions 87 of the regulating member 7 engage with the end surface on the proximal end side of the second area 62. As a result, the regulating member 7 is not further pulled toward the proximal end side.

As shown in FIG. 38, the user further pulls the manipulation wire 230. The clip 2 is separated from the regulating member 7 and pulled toward the proximal end side. The regulating member 7 has the tapered portion 73 formed on the proximal end side of the projected portion 72 grasped by the pair of arms 21. Therefore, when the regulating member 7 is pulled toward the proximal end side, the pair of arms 21 are unlikely to get caught on the regulating member 7.

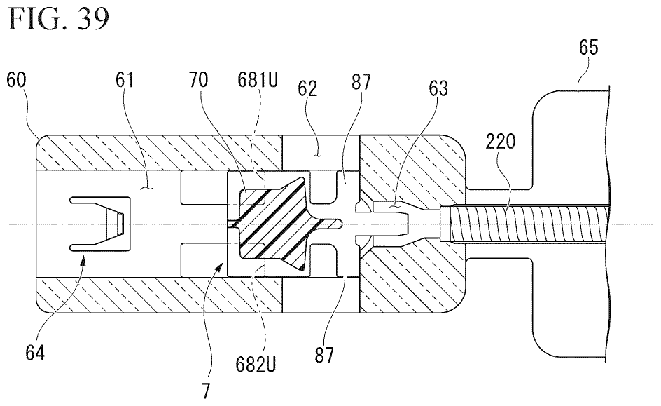

As shown in FIG. 39, the user further pulls the manipulation wire 230. Because the pair of arms 21 do not grasp the regulating member 7, the minimum approach distance between the clip 2 and the pressing member 3 is not regulated. The clip 2 pulled toward the proximal end side is pulled into the internal space of the pressing member 3 and into the sheath 220. The clip 2 may be pulled into the sheath 220 without being pulled into the internal space of the pressing member 3. Thereby, the loading of the clip unit 1 into the clip introduction device 200 is completed. The user releases the compression of the sheath 220 by the compressing portion 65, and pulls out the sheath 220 from the case 6.

### [Method for Storing Clip Unit 1]

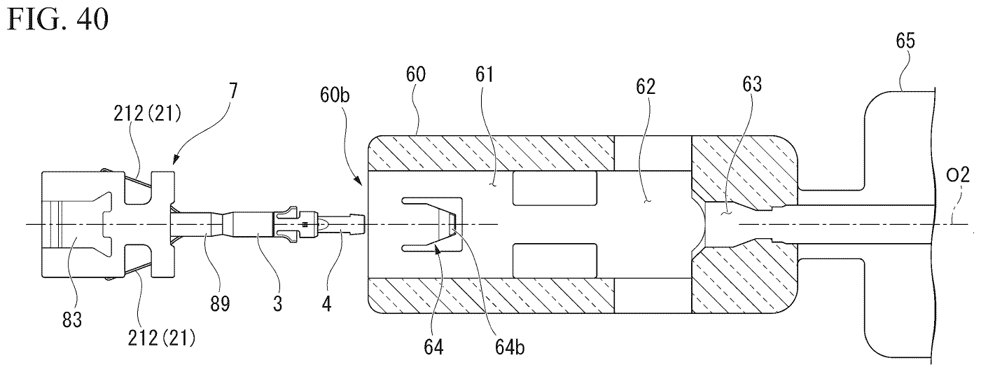

Next, a method for storing the clip unit 1 in the cartridge 5 will be described. FIGS. 40 to 43 are explanatory views of a method for storing the clip unit 1 in the cartridge 5.

As shown in FIG. 40, the user closes the pair of arms 21 with their hands or the like to cause the pair of arms 21 to grasp the regulating member 7. Subsequently, the user inserts the clip unit 1 grasping the regulating member 7 into the storage area 6S of the cartridge 5 from the distal end opening 60b.

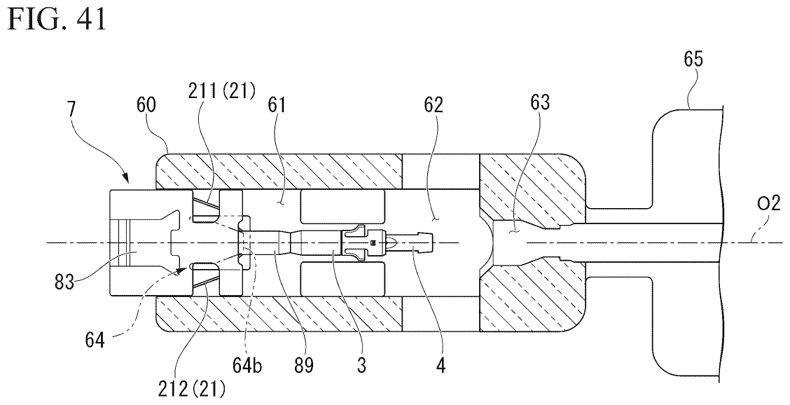

As shown in FIG. 41, the user pushes the regulating member 7 toward the proximal end side to move the clip unit 1 toward the proximal end side. The length W1 of the first area 61 in the width direction W is smaller than the open width W3 of the pair of arms 21 in the open state. Therefore, when the pair of arms 21 grasping the regulating member 7 are inserted into the first area 61, the first arm 211 contacts one end of the distal end opening 60b in the width direction W, and the second arm 212 contacts the other end of the distal end opening 60b in the width direction W. When the user further pushes the regulating member 7 toward the proximal end side, the pair of arms 21 in contact with the distal end opening 60b are further closed and inserted into the first area 61.

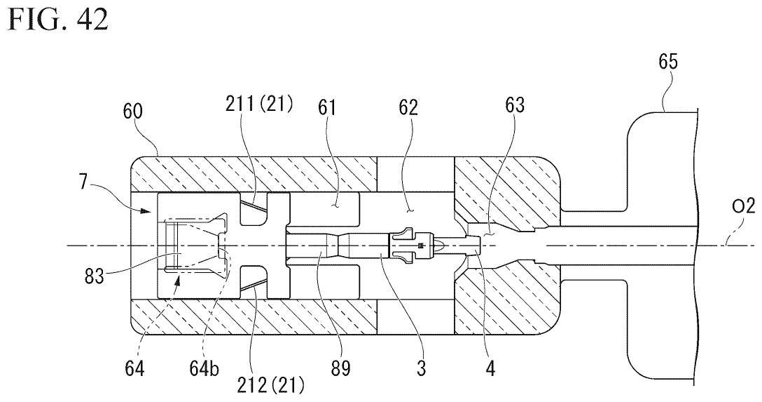

As shown in FIG. 42, when the regulating member 7 moves from the distal end side toward the proximal end side near the locking portion 64 of the case 6, it passes by pushing the locking portion 64 aside toward one side (upper side) in the height direction H. As shown in FIG. 19, the locking portion 64 elastically deforms as a cantilever beam using an end 64c on the distal end side in the longitudinal direction L as a fixed end and an end 64d on the proximal side in the longitudinal direction L as a free end. Therefore, the regulating member 7 moving from the distal end side to the proximal end side easily elastically deforms and pushes aside the locking portion 64.

As shown in FIG. 19, an inclined surface 64s is formed on the distal end side of the locking convex portion 64b in the longitudinal direction L. When the regulating member 7 moving from the distal end side to the proximal end side comes into contact with the locking convex portion 64b of the locking portion 64, the regulating member 7 comes into contact with the inclined surface 64s. Therefore, the regulating member 7 moving from the distal end side to the proximal end side tends to push aside the locking portion 64 toward one side (upper side) in the height direction H.

In addition, even if the regulating member 7 does not have the engaging concave portion 83, the regulating member 7 moves from the distal end side to the proximal end side and therefore can pass while pushing the locking portion 64 aside toward one side (upper side) in the height direction H. However, because the regulating member 7 has the engaging concave portion 83, a distance over which the regulating member 7 passes while pushing aside the locking portion 64 upward is short, making a manipulation easy for the user.

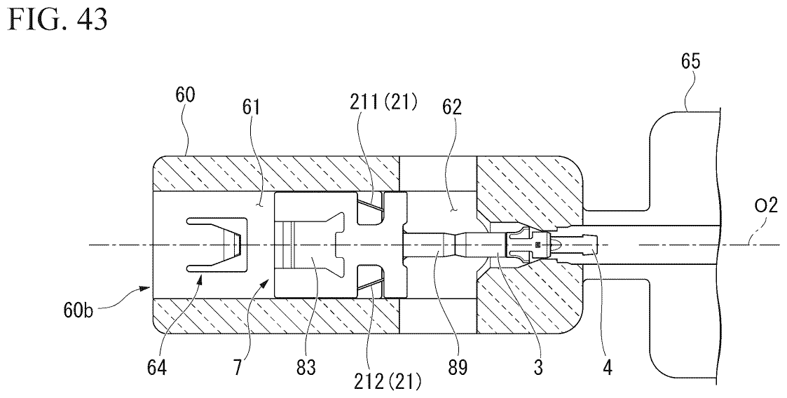

As shown in FIG. 43, after the regulating member 7 passes near the locking portion 64 of the case 6, the locking portion 64 returns to its initial state.

### [Operation and Function of Clip Unit 1]

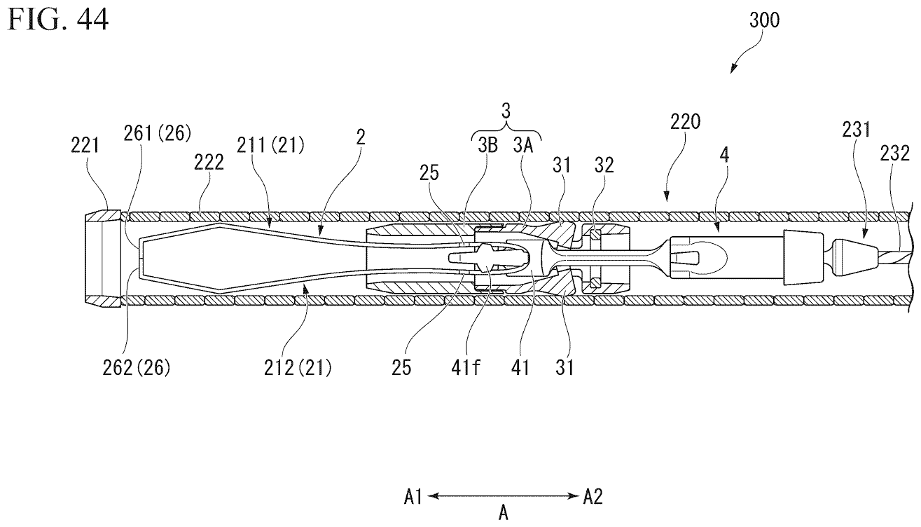

Next, the operation and function of the clip unit 1 will be described with reference to FIGS. 44 to 48.

FIG. 44 is a view showing the clip unit 1 loaded into the clip introduction device 200.

The clip unit 1 is loaded into the clip introduction device 200 using the cartridge 5. The connecting member 4 of the clip unit 1 loaded into the clip introduction device 200 is connected to the arrowhead hook 231 inserted through the inside of the sheath 220. The retractable wings 31 are pressed against the inner circumferential surface of the sheath 220 and are in a retracted state.

The pair of arms 21 of the clip unit 1 that is loaded are pressed against the inner circumferential surface of the sheath 220 and put into the closed state. The engaging portion 25 is located on the distal end side A1 of the fastening member 32, and the pair of arms 21 are not locked in the closed state.

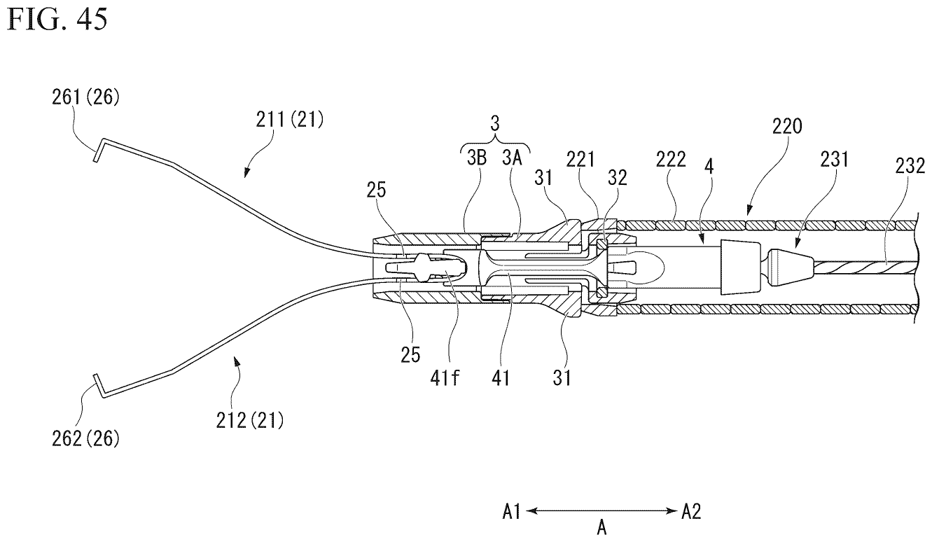

FIG. 45 is a view showing the clip unit 1 introduced into the body.

A surgeon introduces the clip unit 1 loaded into the sheath 220 into the body via the channel of the endoscope. Subsequently, the surgeon advances the slider 242 along the manipulation portion body 241 to advance the arrowhead hook 231. The surgeon advances the clip unit 1 until the retractable wing 31 comes out of the sheath 220. By coming out of the sheath 220, the retractable wing 31 returns from the retracted state to the projected state, which is the basic posture.

When the distal end sides of the pair of arms 21 come out of the sheath 220, the self-expanding force of the pair of arms 21 serves as a restoring force and the clip 2 returns to the open state while moving toward the distal end with respect to the pressing member 3. Even if the pair of arms 21 return to the open state and project most from the pressing member 3, the engaging portion 25 is arranged in the internal space 38 of the pressing member 3.

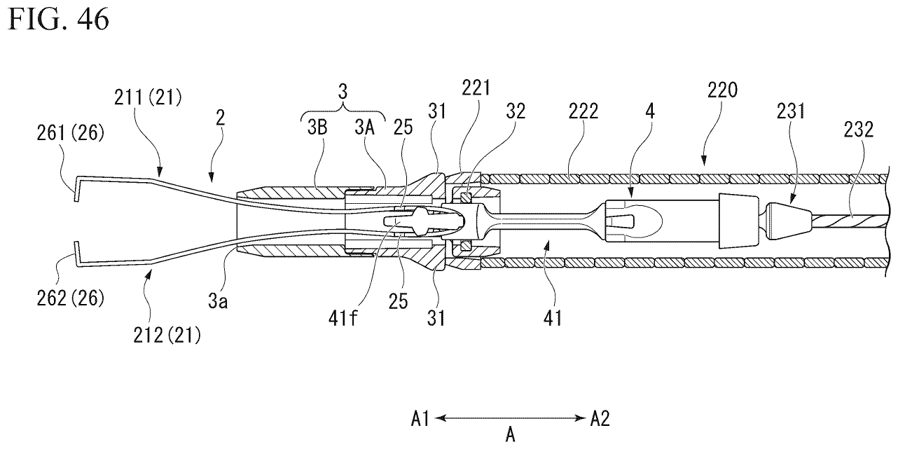

FIG. 46 is a view showing the clip unit 1 with the pair of arms 21 closed.

The surgeon retracts the arrowhead hook 231 by retracting the slider 242 along the manipulation portion body 241. The connecting member 4 connected to the arrowhead hook 231 pulls the clip 2. The pair of arms 21 having a self-expanding force are pulled toward the proximal end side A2, thereby pushing the distal end opening 3a of the pressing member 3 toward the proximal end side A2. The retractable wings 31 in the projected state are engaged with the sheath 220 and are therefore not pulled into the sheath 220. Therefore, the clip 2 pulled by the connecting member 4 is pulled into the pressing member 3.

When the proximal end portion 28 of the clip 2 is pulled toward the proximal end side A2 of the pressing member 3 by the connecting member 4, the pair of arms 21 are pulled into the pressing member 3 and the pair of arms 21 gradually close. When the pulling force on the proximal end portion 28 is released in this state, the clip 2 returns to the open state while moving toward the distal end side A1, using the self-expanding force of the pair of arms 21 as a restoring force. The surgeon can return the pair of arms 21 to the open state to re-grasp the tissue.

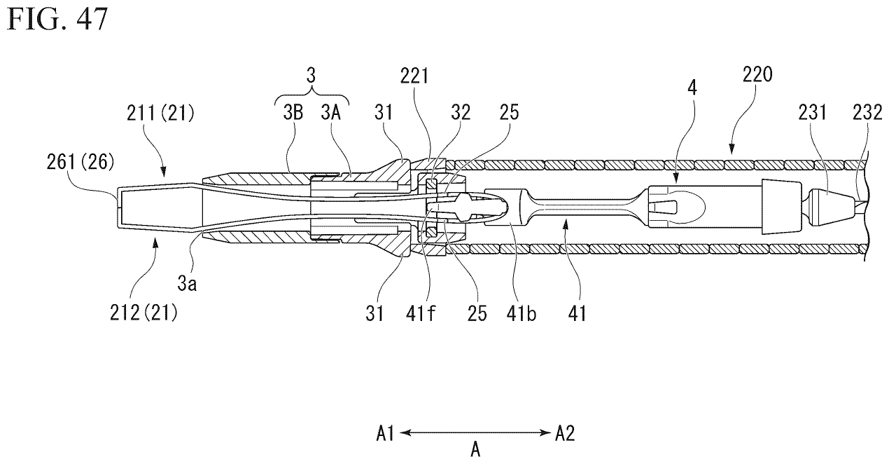

FIG. 47 is a view showing the clip unit 1 with the clip 2 locked.

As the proximal end portion 28 is further pulled toward the proximal end side A2 of the pressing member 3, the engaging portion 25 is pulled toward the proximal end side A2 of the fastening member 32. Because the proximal end side A2 of the engaging portion 25 is formed as an obtuse-angled inclined surface, the engaging portion 25 is easily pulled toward the proximal end side of the fastening member 32. On the other hand, because the distal end side A1 of the engaging portion 25 is formed as an acute-angled inclined surface, if the engaging portion 25 is retracted toward the proximal end side A2 of the fastening member 32, the engaging portion 25 and the fastening member 32 engage with each other. As a result, the movement of the clip 2 toward the distal end side A1 relative to the pressing member 3 is regulated, and the pair of arms 21 are locked in a closed state. If the pair of arms 21 are locked in a closed state, the pair of arms 21 cannot return to an open state.

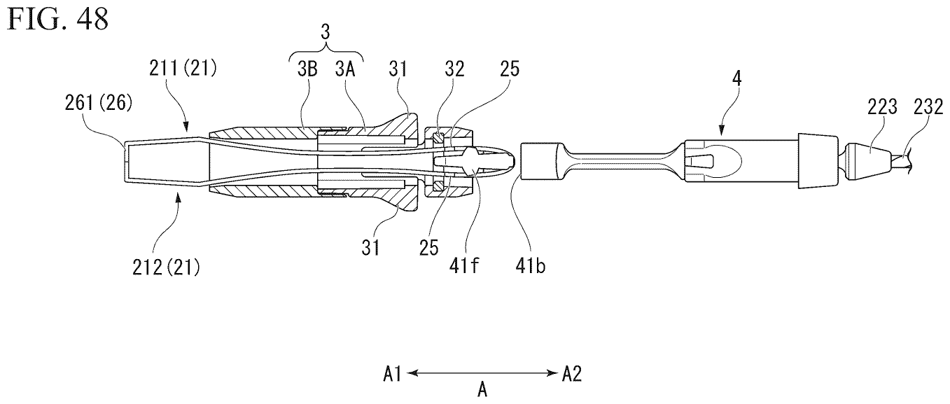

FIG. 48 is a view showing the clip unit 1 from which the clip 2 is separated.

The surgeon further pulls the clip 2. An amount of breaking force of, for example, 20 to 90 newtons (N) due to pulling is applied to the hook 41f, and the breaking portion 41b breaks. The breaking strength of the breaking portion 41b is lower than the breaking strength of the connecting portion body 43. Therefore, the breaking portion 41b breaks instead of the connecting portion body 43.

The surgeon reloads a new clip unit 1 into the clip introduction device 200 using a cartridge or the like.

According to the cartridge system 100 of the present embodiment, when the clip unit 1 is loaded into the clip introduction device 200, the position of the clip unit 1 in the cartridge 5 is unlikely to be misaligned and the clip unit 1 can be easily loaded into the clip introduction device 200.

Although the first embodiment of the present invention has been described above in detail with reference to the drawings, the specific configuration is not limited to the present embodiment and design modifications and the like within the scope of the present invention are also included. Moreover, the constituent elements shown in the above embodiment and the modification examples shown below can be appropriately combined to form a configuration.

### Modification Example 1

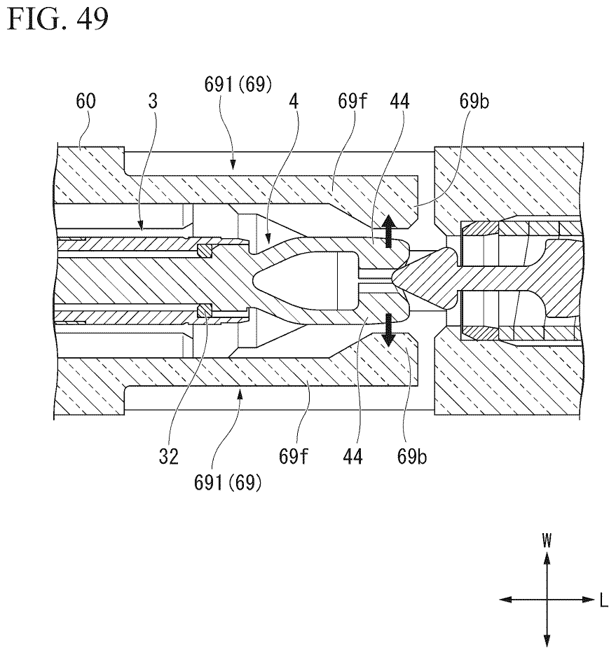

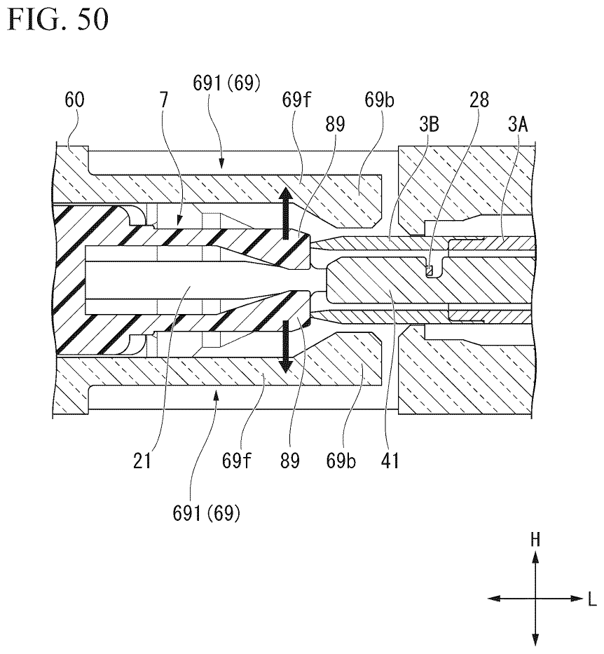

FIGS. 49 and 50 are views showing modification examples of a pair of beam portions 69f.

Second ends 69b of the pair of beam portions 69f may be arranged at positions that engage with the proximal ends of the connecting arms 44. When the second ends 69b are arranged on the proximal end side, the pressing portions 89 of the regulating member 7 that are spread do not engage with the second ends 69b, as shown in FIG. 50. Therefore, the pressing portions 89 are easily spread outward in the height direction H.

### Second Embodiment

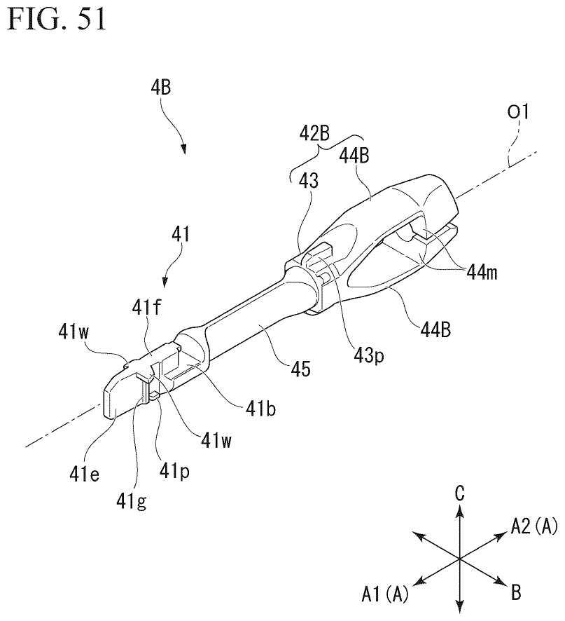

A second embodiment of the present invention will be described with reference to FIGS. 51 to 59. In the following description, constituent elements identical to those already described are denoted by the same reference signs and redundant description will be omitted. A cartridge system 100B according to the second embodiment is the same as the cartridge system 100 according to the first embodiment, except that a connecting member 4B is provided instead of the connecting member 4.

FIG. 51 is a perspective view of the connecting member 4B.

The connecting member 4B is detachably connected to a proximal end portion 28 of a clip 2. Moreover, the connecting member 4B is also detachably connected to an arrowhead hook 231 that passes through a sheath 220. That is, the connecting member 4B connects the clip 2 to the arrowhead hook 231. The connecting member 4B includes a distal end connecting portion 41 provided at the distal end, a proximal end connecting portion 42B provided at the proximal end, and a rod-shaped portion 45 that connects the distal end connecting portion 41 and a proximal end connecting portion 42.

The proximal end connecting portion 42B is an engaging portion that engages with (or connects to) the arrowhead hook portion 231 of the clip introduction device 200. The proximal end connecting portion 42B has a connecting portion body 43 and a connecting arm 44B.

The connecting arms (a pair of legs) 44B are provided at the proximal end of the connecting portion body 43 and are bifurcated. The connecting arm 44B is elastically deformable in an up-down direction C with respect to the connecting portion body 43 and can be opened and closed with respect to the connecting portion body 43. A notch portion 44m that grasps and stores an engaging portion 231a of the arrowhead hook 231 is formed in the connecting arm 44B. The connecting arm 44B can be connected to the arrowhead hook 231 of the clip introduction device 200 in an open state and can maintain the connection with the arrowhead hook 231 of the clip introduction device 200 in a closed state.

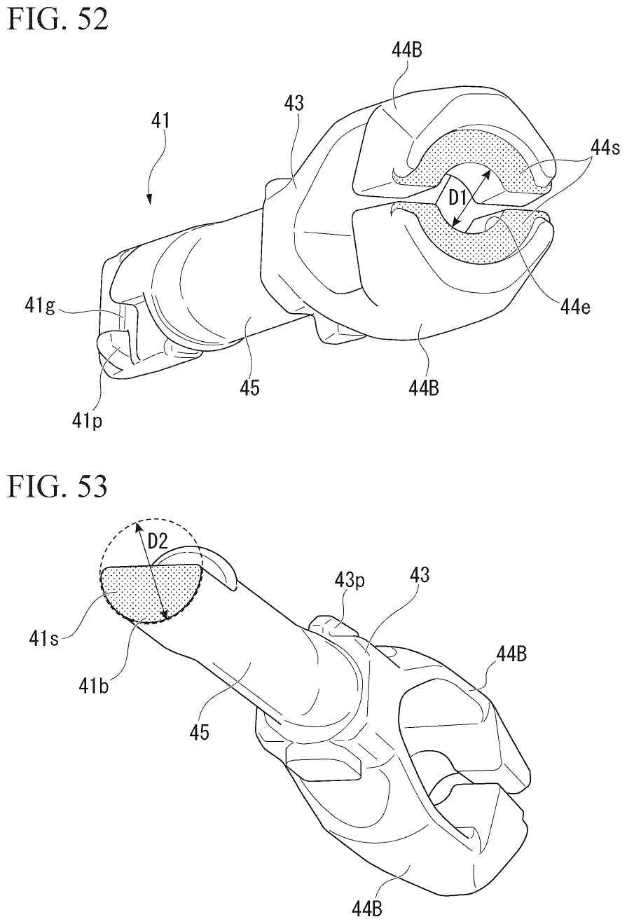

FIG. 52 is a perspective view of the connecting member 4B when seen from the proximal end side A2.

An insertion opening 44e into which the arrowhead hook 231 is inserted is formed on the proximal end side A2 of the connecting arm 44B. A proximal end surface 44s is formed around the proximal end of the insertion opening 44e.

FIG. 53 is a perspective view of the connecting member 4B with a breaking portion 41b broken.

A radial length D2 (maximum possible length) perpendicular to a longitudinal direction A in a breaking surface 41s of the breaking portion 41b is greater than an inner diameter D1 of the insertion opening 44e.

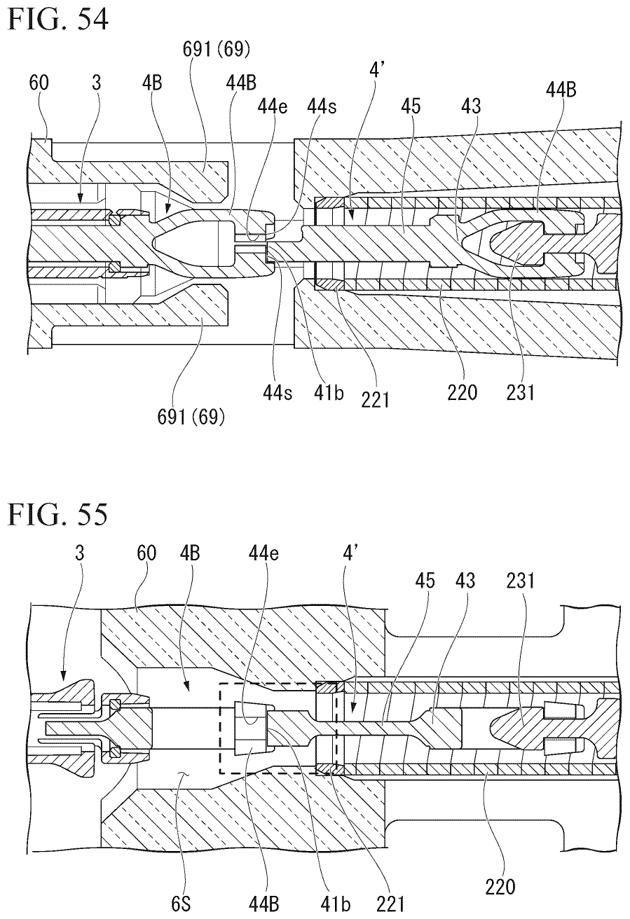

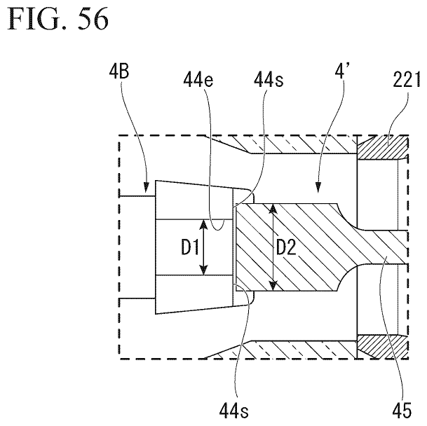

FIGS. 54 and 55 are cross-sectional views showing a connecting member 4′ in which the breaking portion 41b is broken and the distal end side member is separated. FIG. 56 is an enlarged view of the area surrounded by the dotted line in FIG. 55. The connecting member (also called the residue) 4′ from which the distal end side member is separated can be removed from the arrowhead hook 231 by the user. However, there are cases where the user inserts the arrowhead hook 231 into a new cartridge 5 without removing the residue 4′ and attempts to load a new clip unit 1 into the arrowhead hook 231. Even in this case, because the length D2 is greater than the inner diameter D1 of the insertion opening 44e, the breaking surface 41s of the breaking portion 41b abuts against the proximal end surface 44s. As a result, the force in the direction in which the connecting arm 44B is opened is reduced, and the residue 4′ is prevented from being erroneously connected to the connecting arm 44B of the connecting member 4B of the new clip unit 1.

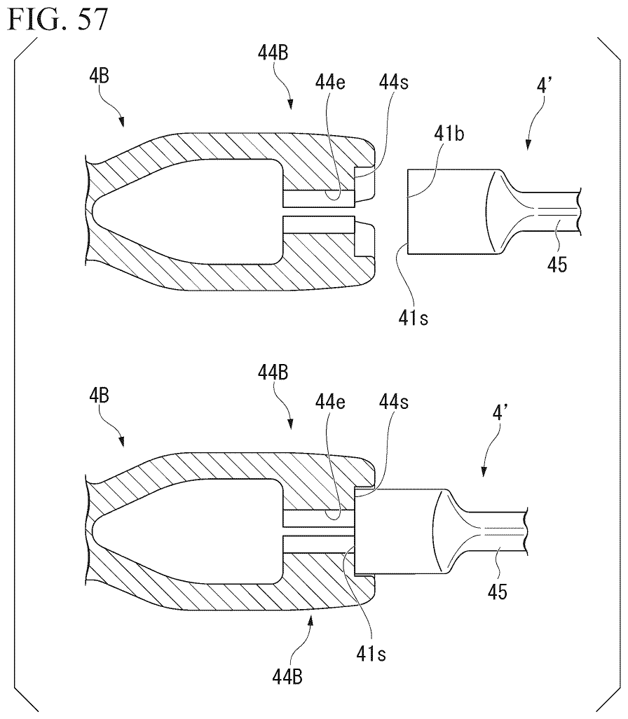

FIGS. 57 to 59 are cross-sectional views showing aspects of the proximal end surface 44s.

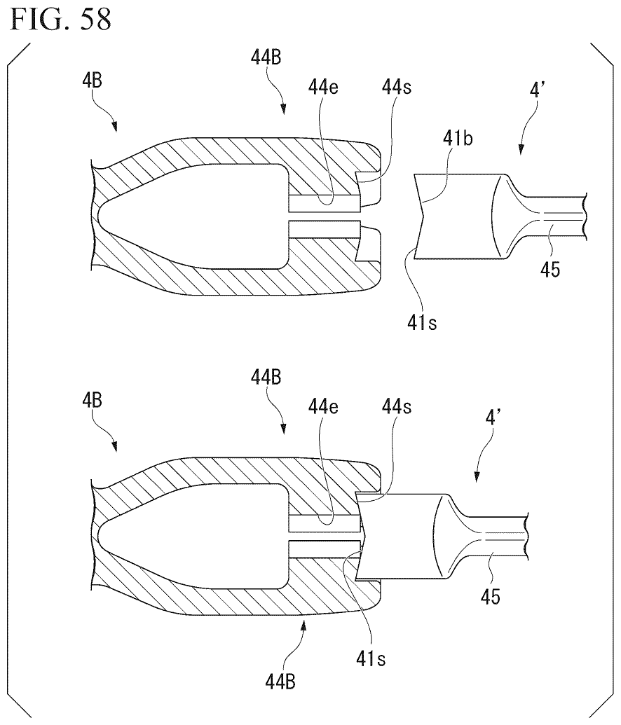

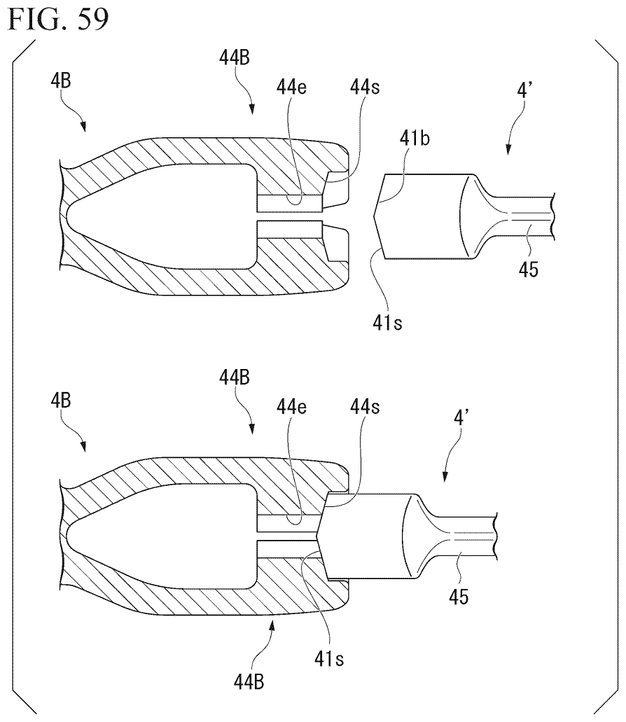

In order to suitably reduce the force acting in the direction in which the connecting arms 44B are opened, the proximal end surface 44s is desirably perpendicular to the longitudinal direction A as shown in FIG. 57. In addition, the proximal end surface 44s may be a surface that is inclined at an angle of about 10 to 20 degrees with respect to the longitudinal direction A as shown in FIGS. 58 and 59.

According to the cartridge system 100B of the present embodiment, when the clip unit 1 is loaded into the clip introduction device 200, the position of the clip unit 1 in the cartridge 5 is unlikely to be misaligned and the clip unit 1 can be easily loaded into the clip introduction device 200. According to the cartridge system 100B of the present embodiment, the residue 4′ is prevented from being erroneously connected to the connecting arm 44B of the connecting member 4B of the new clip unit 1.

Although the second embodiment of the present invention has been described above in detail with reference to the drawings, the specific configuration is not limited to the present embodiment and design modifications and the like within the scope of the present invention are also included. Moreover, the constituent elements shown in the above embodiment and the modification examples shown below can be appropriately combined to form a configuration.

### MODIFICATION EXAMPLES

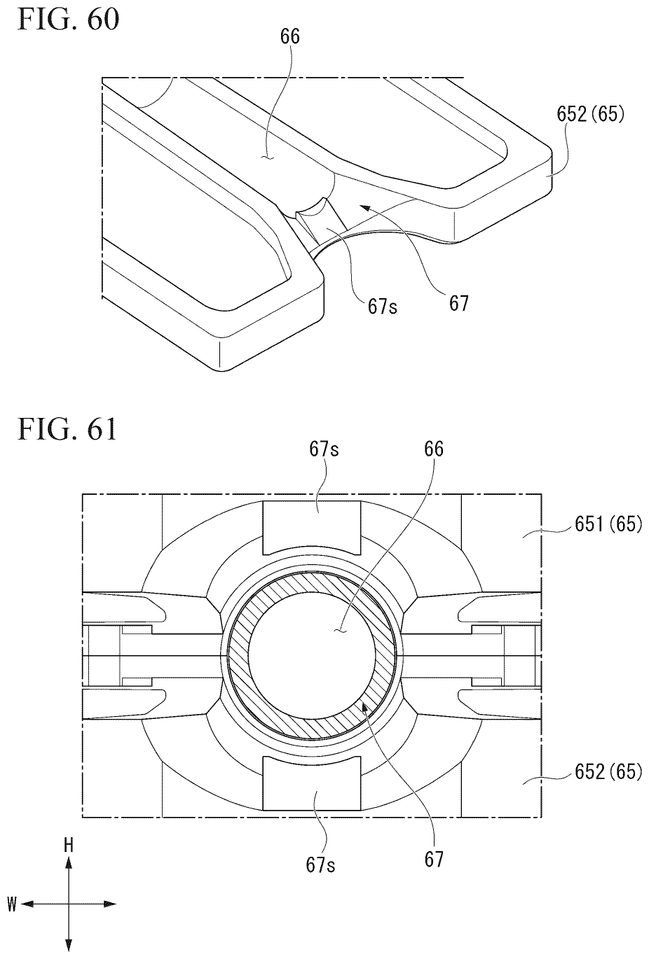

FIGS. 60 and 61 are views showing modification examples of the proximal end opening 67.

The compressing portion 65 (the first compressing portion 651 and the second compressing portion 652) of the case 6 may have a contact surface 67s configured to project in the height direction H in the proximal end opening 67. When the user compresses the sheath 220 with the compressing portion 65, the contact surface 67s comes into contact with the sheath 220, and the sheath 220 can be fixed to the case 6 more reliably.

What is claimed is:

1. A cartridge system comprising:
a clip unit having an arm; and
a case housing the clip unit,
wherein the case has a cantilever beam extending in a longitudinal direction of the case.

2. The cartridge system according to claim 1,
wherein the cantilever beam has a first end fixed to the case and a second end spaced apart from the case, and
wherein the first end is arranged on a distal end side of the second end in the longitudinal direction.

3. The cartridge system according to claim 2, wherein the second end projects further inward from the case than the first end.

4. The cartridge system according to claim 1,
wherein the case has a recess, and
wherein the cantilever beam is housed in the recess.

5. The cartridge system according to claim 1,
wherein the clip unit is configured to be attachable to an applicator,
wherein the case has a storage area in which the clip unit is stored and an insertion area into which at least a part of the applicator is inserted,
wherein the cantilever beam is arranged on a proximal end side of the storage area in the longitudinal direction, and
wherein the cantilever beam is arranged on a distal end side of the insertion area in the longitudinal direction.

6. The cartridge system according to claim 1, comprising a slider housed in the case,
wherein the clip unit and the slider are configured to be movable inside the case, and
wherein the cantilever beam is configured to be able to come into contact with the clip unit or the slider.

7. The cartridge system according to claim 1,
wherein the cantilever beam has a first cantilever beam and a second cantilever beam,
wherein the cantilever beam is deformable between a first state in which a length between the first cantilever beam and the second cantilever beam in a height direction is a first length and a second state in which the length between the first cantilever beam and the second cantilever beam in the height direction is a second length longer than the first length,
wherein the first length is smaller than an outer width of the clip unit in the height direction, and
wherein the second length is larger than the outer width of the clip unit in the height direction.

8. The cartridge system according to claim 7,
wherein the first cantilever beam and the second cantilever beam each have a first end fixed to the case and a second end spaced apart from the case, and
wherein, when the cantilever beam is in the second state, the second end is arranged in a range not exceeding an outer width of the case in the height direction.

9. The cartridge system according to claim 1,
wherein the clip unit is configured to be movable inside the case in the longitudinal direction, and
wherein the cantilever beam is configured to regulate movement of the clip unit.

10. The cartridge system according to claim 9, wherein the cantilever beam regulates swinging of the clip unit in a direction intersecting the longitudinal direction.

11. The cartridge system according to claim 9, wherein the clip unit has a tube supporting the arm and a slider supporting the tube.

12. A cartridge capable of housing a clip unit having an arm, the cartridge comprising:
a case housing the clip unit,
wherein the case has a cantilever beam extending along a longitudinal direction of the case.

13. The cartridge according to claim 12,
wherein the cantilever beam has a first end fixed to the case and a second end spaced apart from the case, and
wherein the first end is arranged on a distal end side of the second end in the longitudinal direction.

14. The cartridge according to claim 13, wherein the second end projects further inward from the case than the first end.

15. The cartridge according to claim 12,
wherein the case has a recess, and
wherein the cantilever beam is housed in the recess.

16. The cartridge according to claim 12,
wherein the clip unit is configured to be attachable to an applicator,
wherein the case has a storage area in which the clip unit is stored and an insertion area into which at least a part of the applicator is inserted,
wherein the cantilever beam is arranged on a proximal end side of the storage area in the longitudinal direction, and
wherein the cantilever beam is arranged on a distal end side of the insertion area in the longitudinal direction.

17. The cartridge according to claim 12,
wherein the cantilever beam has a first cantilever beam and a second cantilever beam,
wherein the cantilever beam is deformable between a first state in which a length between the first cantilever beam and the second cantilever beam in a height direction is a first length and a second state in which the length between the first cantilever beam and the second cantilever beam in the height direction is a second length longer than the first length,
wherein the first length is smaller than an outer width of the clip unit in the height direction, and
wherein the second length is larger than the outer width of the clip unit in the height direction.

18. The cartridge according to claim 17,
wherein the first cantilever beam and the second cantilever beam each have a first end fixed to the case and a second end spaced apart from the case, and
wherein, when the cantilever beam is in the second state, the second end is arranged in a range not exceeding an outer width of the case in the height direction.

19. The cartridge according to claim 12,
wherein the clip unit is configured to be movable inside the case in the longitudinal direction, and
wherein the cantilever beam is configured to regulate movement of the clip unit.

20. The cartridge according to claim 19, wherein the cantilever beam regulates swinging of the clip unit in a direction intersecting the longitudinal direction.
